SEQUENCE LISTING (7 sequences)

SEQ ID NO: 1 - DNA, 24 bp, Artificial
  PCR primer
ggagacagaagaggagaagattcg

SEQ ID NO: 2 - DNA, 19 bp, Artificial
  PCR primer
tgcccagctgcatatggtt

SEQ ID NO: 3 - DNA, 28 bp, Artificial
  oligo probe
tccatggtctggttctccaaaatgtcca

SEQ ID NO: 4 - DNA, 26 bp, Artificial
  PCR primer
gtctgcttttacatctgaacctctgt

SEQ ID NO: 5 - DNA, 22 bp, Artificial
  PCR primer
agccctgagcagtcttcagaga

SEQ ID NO: 6 - DNA, 28 bp, Artificial
  PCR primer
actctcacacccagatgctgcttcacct

SEQ ID NO: 7 - protein, 13 aa, Artificial
  N-terminal peptide
ALDTNYCFSSTEK

(19) United States
(12) Patent Application Publication
(10) Pub. No.: US 2011/0245213 A1
(43) Pub. Date: Oct. 6, 2011

(54) TREATMENT

(75) Inventors: Niamh O'Kennedy, Berkshire (GB); Richard Mithen, Norwich (GB)

(73) Assignees: PROVEXIS NATURAL PRODUCTS LIMITED, Berkshire (GB); PLANT BIOSCIENCE LIMITED, Norwich (GB)

(21) Appl. No.: 13/001,396

(22) PCT Filed: Jun. 25, 2009
(86) PCT No.: PCT/GB2009/001593

§ 371 Date: Dec. 23, 2010

(30) Foreign Application Priority Data
Jul. 1, 2008 (GB) 0811992.7

(51) Int. Cl. A61K 31/26; C07C 331/16; C07C 331/20; A61K 31/56; A61K 31/352; A61P 29/00

(52) U.S. Cl. 514/171; 558/17; 514/514; 514/515; 514/456

(57) ABSTRACT

The present invention concerns compositions that may be used in the prevention or treatment of medical conditions characterised by having an inflammatory component. The compositions comprise a therapeutically effective amount of an isothiocyanate (ITC). The composition may comprise further anti-inflammatory agents (e.g. plant-derived polyphenols).

The present invention relates to the treatment of conditions characterised by have an inflammatory component and in particular treatment of extracts derived from edible plants and active agents that are derivable from such plants.

A number of medical conditions are characterised by have an inflammatory component which may manifest as inappropriate secretion of inflammatory mediators (e.g. highly toxic reactive oxygen intermediates (ROIs) or granule enzymes or cytokines) from leukocytes, platelets or endothelial cells into an affected tissue. Inflammation is a major factor contributing to the development and pathology of many cancers (for example bowel cancers, prostate cancer and leukaemias), and is a characteristic of diabetes mellitus (Types 1 and 2) and atherosclerotic diseases. Examples of other conditions with inflammatory characteristics include, but are not limited to: Inflammatory Bowel Disease (IBD) Rheumatoid Arthritis (RA), Behcet's Disease, ANCA-associated vasculitis, systemic vasculitis, cystic fibrosis, asthma, dermatitis and psoriasis.

Inflammatory bowel disease (IBD) is a term used to describe idiopathic, chronic inflammation of the gastrointestinal tract and includes two main phenotypes: Crohn's disease (CD) and ulcerative colitis (UC). Crohn's disease is typified by granulomatous inflammation affecting any part of the gastrointestinal tract but particularly the ileocecal area. Ulcerative colitis is colon-specific and is associated with extensive epithelial damage, crypt abscesses and abundant mucosal neutrophils. Patients with extensive UC or colonic Crohn's disease have an approximately ten-fold increased risk of developing colorectal cancer, which represents the major cause of IBD-associated mortality. Given the debilitating nature of IBD, as well as IBD-associated mortality, there is a need to provide new and improved treatments for these conditions.

By way of further example, Rheumatoid Arthritis (RA) is an inflammatory condition characterised by inflamed synovial joints that lead to tissue damage and, ultimately, joint destruction. The potential to decrease inflammatory damage is attractive. However current therapies for RA are inadequate, both in their ability to adequately suppress disease activity and their unacceptable side effects. Treatment today can be considered as traditional (conventional) therapy and biologic therapy. “Traditional” drugs were, on the whole, discovered by serendipity, where a drug developed for a totally different condition was also found to be of benefit in RA. Biologic therapies are expensive to manufacture and manufacturing capacity for biologics cannot keep up with demand.

Isothiocyanates (ITC) are organic molecules, derivable from plants, which comprise the chemical group ⁻N═C═S. ITCs are formed by substituting sulphur for oxygen in the isocyanate group. Allyl isothiocyanate is an example of an ITC found in mustard oil that is responsible for its pungency.

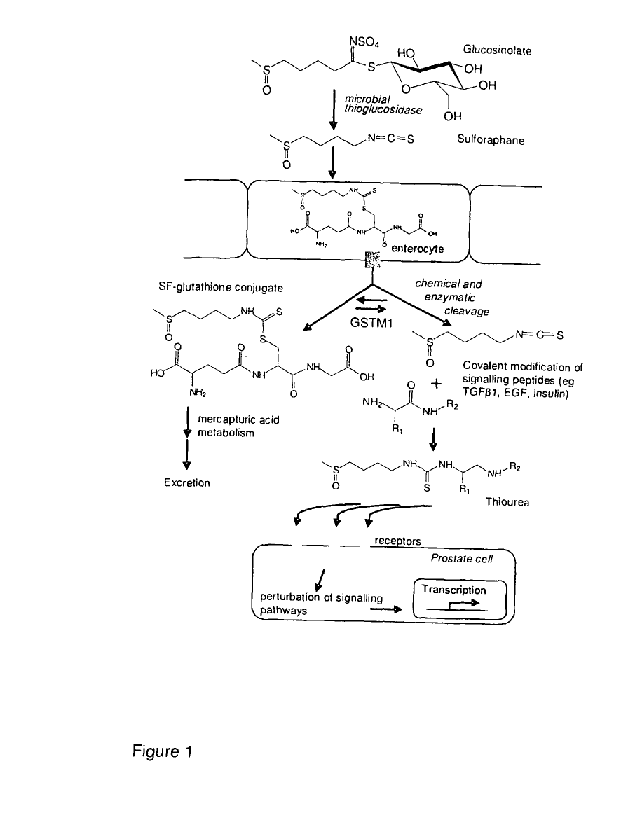

Plants of the genus brassica can be rich in ITCs. For instance, broccoli accumulates 4-methylsulphinylbutyl and 3-methylsulphinylpropyl glucosinolates in its florets. These glucosinolates are converted to the ITCs: sulforaphane (SF), erucin (ER) and iberin (IB), respectively, either by plant thioglucosidases (‘myrosinases’) following tissue damage or, if the myrosinases have been denatured by cooking or blanching prior to freezing, by microbial thioglucosidases in the colon of a subject that has consumed the vegetable (see FIG. 1). SF and IB are passively absorbed by enterocytes, conjugated with glutathione and transported into the systemic circulation to be metabolized via the mercapturic acid pathway and excreted predominantly as N-acetylcysteine conjugates in the urine. Following broccoli consumption, 45% of SF in the plasma occurs as free SF, as opposed to thiol conjugates, and the peak concentration of SF and its thiol conjugates is less than 2 μM, falling to low (nM) levels within a few hours.

ITCs, such as phenethyl isothiocyanate (PEITC) and SF, have been shown to inhibit carcinogenesis and tumorigenesis and as such are useful chemopreventive agents against the development and proliferation of cancers. They may work on a variety of levels. Most notably, they have been shown to inhibit carcinogenesis through inhibition of cytochrome P450 enzymes, which oxidise compounds such as benzo[a]pyrene and other polycyclic aromatic hydrocarbons (PAHs) into more polar epoxy-diols which can then cause mutation and induce cancer development. PEITC has also been shown to induce apoptosis in certain cancer cell lines, and in some cases, is even able to induce apoptosis in cells that are resistant to some currently used chemotherapeutic drugs.

There remains a need to develop new and improved compositions that are useful in the prevention or treatment of medical conditions with an inflammatory component and it is an object of the present invention to address this need.

According to a first aspect of the present invention, there is provided a composition for use in the prevention or treatment of medical conditions characterised by having an inflammatory component comprising a therapeutically effective amount of an isothiocyanate (ITC) or a precursor thereof.

According to a second aspect of the present invention, there is provided a therapeutically effective amount of an isothiocyanate (ITC), or a precursor thereof, for use as a medicament for the prevention or treatment of medical conditions characterised by having an inflammatory component.

According to a third aspect of the present invention, there is provided a method for the treatment of medical conditions characterised by having an inflammatory component comprising administering to a subject in need of such treatment a therapeutically effective amount of ITC or a precursor thereof.

By “medical conditions characterised by having an inflammatory component” we mean any medical condition at least partially characterised by inappropriate secretion of inflammatory mediators (e.g. highly toxic reactive oxygen intermediates (ROIs) or granule enzymes or cytokines). Examples of such conditions include, but are not limited to: Inflammatory Bowel Disease (IBD) Rheumatoid Arthritis (RA), Behcet's Disease, ANCA-associated vasculitis, systemic vasculitis, cystic fibrosis, asthma, dermatitis and psoriasis. Inflammation is also major factor contributing to the development and pathology of many cancers. Therefore cancers (for example bowel cancers, prostate cancer and leukaemias) with an inflammatory component are also included within the definition of medical conditions. Inflammation is also a characteristic of diabetes mellitus (Types 1 and 2) and atherosclerotic diseases and these conditions are also encompassed by the term.

By “isothiocyanate (ITC)” we mean organic molecules, which may be derivable from plants, that comprise the chemical group ⁻N═C═S. ITCs are formed by substituting sulphur for oxygen in an isocyanate group. By “ITC” we also include glucosinolate precursors that may be easily metabolised to form ITCs. Preferred ITCs are derivable from Brassica (e.g. from broccoli or rocket) and include sulforaphane (SF), iberin (IB) and erucin (ER-4-methylthiobutyl isothiocyanate). Preferred ITCs, such as SF and IB, do not have the pungent flavour qualities associated with some dietary ITCs (e.g. Allyl isothiocyanate from mustards).

By a “precursors thereof” we mean phytochemicals, which may be produced naturally in plants, that may be converted to an active ITC. In particular we mean glucosinolates and glucosinolate derivatives (e.g. indole derivatives of glucosinolates) that may be found in brassica such as rocket or broccoli which may be converted to ITCs by plant thioglucosidases (e.g. following plant tissue damage) or by microbial thioglucosides in the colon of a subject that has consumed a composition containing the precursors.

### Sources of ITCs

It will be appreciated that ITCs used according to the invention may be chemically synthesised or may be derived from any natural or unnatural (e.g genetically modified microorganisms or cell lines) sources.

However, according to a preferred embodiment of the invention the ITC, or precursor thereof, is derived from plants and preferably plants of the family Brassicaceae or Capparaceae. It is preferred that the ITC is derivable from a plant of the genus Brassica and more preferred that the ITC is derived from mustard, broccoli or rocket. It is most preferred that the ITC is derivable from rocket (e.g Eruca and Diplotaxis spp)

It will be appreciated that when ITC is derived from Brassica that the compounds may be isolated and purified from plants. Such purification may be desirable under some circumstance (e.g. when pharmaceutical grade purity is needed of the active ITC). However in many circumstances, such as in food, drink or nutraceutical products, it may be preferred to produce a plant extract that is enriched in ITC.

Plant extracts represent an important embodiment of the invention and according to a fourth aspect of the present invention, there is provided a plant extract for use in the prevention or treatment of medical conditions characterised by having an inflammatory component wherein the plant extract is enriched in ITC or a precursor thereof.

According to a fifth aspect of the present invention, there is provided a plant extract enriched in ITC or a precursor thereof for use as a medicament for the prevention or treatment of medical conditions characterised by having an inflammatory component.

According to a sixth aspect of the present invention, there is provided a method for the prevention or treatment of medical conditions characterised by having an inflammatory component comprising administering to a subject in need of such prevention or treatment a therapeutically effective amount of a plant extract enriched in ITC or a precursor thereof.

By “a plant extract that is enriched in ITC” we mean that a plant has be processed such that ITC, and precursors thereof, are maintained in the extract in active form. The plant may be treated such that the concentration of the ITC in the extract is increased when compared to the concentration in unprocessed plants. Alternatively the extract may contain ITC or a precursor thereof, which is substantially active, that may be at about the same concentration (or even less if substantially diluted) as found in the untreated plant.

Although we do not wish to be bound by any hypothesis the inventors believe that compounds, extracts and compositions according to the invention are useful in the treatment and prevention of medical conditions characterised by have an inflammatory component based upon their understanding of this scientific field and particularly in view of the work presented in Example 1. The inventors have established that ITCs bind to cytokines; promote anti-inflammatory signalling pathways (e.g. Smad activation); and reduce the expression of the pro-inflammatory cytokine IL-6. This demonstrates that ITCs and precursors thereof, and plant extracts enriched in ITCs and/or precursors thereof, are useful for modulating conditions according to the invention.

### Preparation of Plant Extracts Enriched in ITC

It is preferred that the plant extracts enriched in ITC are based on plants of the family brassicaceae or Capparaceae (glucosinolate containing plants). Preferrably the plant extract is from the family brassicaceae and genus Brassica.

Preferred extracts are derived from plants such as mustard, broccoli or rocket. It is most preferred that the plant extract is rocket (e.g Eruca and Diplotaxis spp) extract.

It will be appreciated that a crude plant extract may be prepared by crushing plant leaves, stems or seeds (preferably at temperatures up to 25° C.). The crushed leaves may then be homogenised in an aqueous solution to form a liquid plant extract according to the invention. Vegetable solids may be pelleted by centrifuging and the supernatant (containing ITC) may be used as an extract according to the invention.

It is preferred that the plant extract is prepare from fresh leaves from young plants (e.g. rocket plants of 28-42 days) and/or from young sprouts (e.g. rocket plants up to 14 days).

Preferred extracts comprising ITCs are derived from fresh leaves or young sprouts that are dried (e.g. by air drying or by snap freezing and freeze drying). The dried material may then be processed by:
(a) Milling to a fine powder.
(b) Making a suspension of this milled powder by mixing powder with water or other aqueous solution to give a mixture with a minimum of 10% solids and a maximum 50% solids.
(c) ITCs are then extracted from the suspension. This may be achieved using a counter-current extractor, equipped with a vapour trap to retain volatiles extracted into solution, or a Soxhlet-type extractor operating under reduced pressure and fitted with a reflux condenser. Extraction should proceed until a minimum of 50%, and preferably >70%, of the native glucosinolates from the rocket has been converted to ITCs by the action of native enzymes.
(d) Once extraction is complete, solids can be removed from the suspension by centrifugal separation or decanting. The ITC-rich supernatant can be deproteinated by chemical or enzymatic means, or by filtration (e.g. ultrafiltration), and concentrated by low-temperature high vacuum evaporation, or by removal of water by reverse osmosis.
(e) The final extract can be stored frozen as a liquid or spray-dried to give a powder, or encapsulated (e.g. in a fat matrix, or in a polysaccharide matrix, or in a polymer matrix) to enhance stability.

In another preferred embodiment seeds (e.g. mustard or rocket seeds) can be used as the starting material. In the case of seeds, air drying is sufficient preparation, and the dry seeds can then be crushed (for example using a sealed press) in the presence of water to give a high solids mash (e.g. between 75% and 90% solids). Crushing should proceed until a homogenous mash is formed; thereafter the extraction can proceed as described above (see (c)-(e)).

It will be appreciated that a mixture of sprouts/leaves/seeds may be used as the starting material, to ensure an ITC extract containing a wide range of structures is prepared. Leaves and sprouts contain higher levels of 4-mercaptobutyl GLS than seeds, which are higher in 4-methylthiobutyl GLS.
An alternative preferred plant extract according to the invention may be enriched in glucosinolate (i.e. an ITC precursor according to the invention). Starting materials may be seeds, sprouts or leaves (preferably dried prior to extraction) as described above. A suspension of dried, milled starting material may then be made in an ethanolic solution (e.g. 70%-85% ethanol), to give a mixture with minimum 10% solids, maximum 50% solids. The ethanol used is preferrably food-grade. The ethanol solution is then heated in a reactor (preferably a counter-current continuous extractor or a Soxhlet-type extractor equipped with condensers to catch volatiles) at about 70° C. until between 70% and 90% of the native glucosinolates have been extracted into ethanolic solution. Solids may then be removed from the suspension by centrifugal separation or decanting and the ethanol removed from the supernatant by, for example, evaporation under reduced pressure, or by reverse osmosis (using diafiltration) after first diluting the supernatant to <40% ethanol. The final solution should contain <5% ethanol. This glucosinolate-rich solution can either be stored frozen, or can be spray-dried to give an ethanol-free powder. To convert the glucosinolates to ITCs, the glucosinolate-rich extract may be dissolved in water at 20-30° C., and the conversion should be carried out by adding myrosinase enzyme, either in purified form or as part of a crude rocket-seed/mustard-seed mash. The mixture should be incubated until a minimum of 50%, and preferably >70%, of the native glucosinolates have been converted to ITCs. Solid material and protein may be removed from the ITC-rich solution by filtration (e.g. microfiltration or ultrafiltration), and the extract can then be concentrated as previously described.

### Formulations of Compositions According to the Invention

Clinical needs may dictate that the plant extracts discussed above may need to be used substantially “neat” or even simply diluted in an aqueous solution. When this is the case the supernatant (whether diluted or not) may be mixed with a number of other agents that may be added for nutritional reasons, medical reasons or even for the purposes of adjusting the palatability of the extract for consumption by the subject being treated.

For instance, the extract may be formulated with a diary product (e.g. milk, a milk shake or yoghurt) or a fruit juice (e.g. grape juice, orange juice or similar) to produce a palatable drink/beverage with the added benefit that it contains ITCs, or precursors thereof, and therefore will be highly suitable as a refreshment for sufferers of inflammatory conditions.

Alternatively, the plant extract may be included in a nutritional liquid for enteral feeding. For instance, the supernatant may be mixed with saline or an aqueous solution (other vitamins, minerals and nutrients may be included) for enteral feeding of subjects.

It is preferred that liquids comprising ITCs have a concentration of ITC of between 1 and 1000 μM and preferably between 10 and 100 μM.

Compositions according to the invention may be formulated as powders, granules or semisolids for incorporation into capsules. For presentation in the form of a semisolid, the ITC, or vegetable extract enriched in ITC, can be dissolved or suspended in a viscous liquid or semisolid vehicle such as a polyethylene glycol, or a liquid carrier such as a glycol, e.g. propylene glycol, or glycerol or a vegetable or fish oil, for example an oil selected from olive oil, sunflower oil, safflower oil, evening primrose oil, soya oil, cold liver oil, herring oil, etc. This may then be filled into capsules of either the hard gelatine or soft gelatine type or made from hard or soft gelatine equivalents, soft gelatine or gelating-equivalent capsules being preferred for viscous liquid or semisolid fillings.

Powders comprising ITC, or vegetable extract enriched in ITC, according to the invention are particularly useful for making pharmaceutical or nutritional products that may be used to prevent or treat conditions at least partially characterised by inflammation.

Freeze-drying or spray drying represent preferred methods for producing a powder according to the invention. Spray drying results in free-flowing granular powder mixes with good flow properties and quick dissolving characteristics.

It will be appreciated that spray-dried or freeze-dried powder produced by the protocols discussed above represent preferred powdered compositions according to the invention. A preferred powder is derived from a reconstituted vegetable extract enriched in ITC which is subsequently freeze-dried or spray-dried.

Powdered compositions may be reconstituted as a clear/translucent low viscosity drink/beverage. Reconstitution may be into water or dairy or fruit juices as discussed above. It will be appreciated that the powder may be packaged in a sachet and reconstituted as a drink by a subject when required or desired.

Powder mixes represent preferred embodiments of the invention. Such mixes comprise powdered ITC, or powdered vegetable extract enriched in ITC, mixed with further ingredients. Such ingredients may be added for nutritional or medical reasons or for improved palatability. The powdered composition may be mixed with granulated sugars of varying particle sizes to obtain free-flowing powder mixes of varying sweetness.

Alternatively natural sweeteners or artificial sweeteners (e.g. aspartame, saccharin and the like) may be mixed with the powdered compositions for reconstitution as a low calorie/reduced calorie sweetened drink. The powder mix may comprise a mineral supplement. The mineral may be any one of calcium, magnesium, potassium, zinc, sodium, iron, and their various combinations.

Powder mixes may also contain buffering agents such as citrate and phosphate buffers, and effervescent agents formed from carbonates, e.g. bicarbonates such as sodium or ammonium bicarbonate, and a solid acid, for example citric acid or an acid citrate salt.

ITC, or vegetable extract enriched in ITC can be presented as food supplements or food additives, or can be incorporated into foods, for example functional foods or nutriceuticals. Such products may be used as staple foods as well as under circumstances where there may be a clinical need.

The powders may be incorporated in to snack food bars for example fruit bars, nut bars and cereal bars. For presentation in the form of snack food bars, the powder can be admixed with any one or more ingredients selected from dried fruits such as sundried tomatoes, raisins and sultanas, ground nuts or cereals such as oats and wheat.

It will be appreciated that compositions according to the invention may advantageously be formulated as a pharmaceutical product for use as a medicament (requiring a prescription or otherwise).

Powdered compositions or concentrated liquid extracts enriched in ITC may also be incorporated into tablets, lozenges, sweets or other food-stuffs for oral ingestion. It will also be appreciated that such powdered compositions or concentrated liquid extracts may be incorporated into slow-release capsules or devices which may be ingested and are able to release ITC into the intestines over a long period of time.

Compositions according to the invention may also be microencapsulated. For instance encapsulation may be by calcium-alginate gel capsule formation. Kappa-carrageenan, gellan gum, gelatin and starch may be used as excipients for micro-encapsulation.

### Combination Therapies

It will be appreciated that compositions, medicaments and extracts according to the present invention may be used alone or alternatively may also be mixed with other extracts, compositions or compounds (provided those compounds do not inhibit the anti-inflammatory properties of ITC according to the invention). Accordingly the present invention also encompasses compositions comprising effective amounts of the ITC and others active agents.

Compositions, medicaments and extracts according to the present invention may be combined with known therapeutic agents for treating medical conditions according to the invention. As such the composition may be used in a very effective combination therapy. It will be appreciated that the composition in solution may act as an ideal vehicle for other therapeutic agents for treating the conditions.

Examples of other active agents that may be combined with compositions, medicaments and extracts according to the present invention include non-steroidal anti-inflammatory drugs (NSAIDs) and corticosteroids. The compositions, medicaments and extracts can also be combined with other therapeutic agents that are targeted at a specific condition. For instance, when RA is prevented or treated according to the invention a combination therapy may include orally active “disease-modifying” anti-rheumatic drugs (DMARDs) or biologics used to treat RA (e.g. anti-cytokine antibodies and cytokine receptor antagonists).

ITC, or plant extracts enriched in ITC may also be included in combination/synbiotic therapies that include a probiotic portion.

In a most preferred embodiment of the invention the compositions comprising ITCs or plant extracts enriched in ITC and precursors thereof, may be combined with a polyphenol. The inventors have found to their surprise that ITCs and polyphenols are very effective in a combination therapy for treating medical conditions according to the invention (see Example 4). Compositions comprising ITCs and polyphenols, or plant extracts enriched in ITCs and polyphenols, represent an important feature of the present invention. Therefore according to a seventh aspect of the invention there is provided a composition comprising a therapeutically effective amount of an ITC and a polyphenol. Such compositions are particularly useful for treating the medical conditions discussed herein.

The polyphenol is preferably derivable from a fruit and more preferably from a fruit skin or fruit seed. It is preferred that the fruit is a Vinus spp. Therefore a preferred source of polyphenol could be grape skins or grape seeds and a most preferred source of polyphenols is a grape juice that has been processed such that it retains polyphenols from grape skins and seeds within the juice.

The polyphenol is preferably procyanadin. Alternatively the polyphenol may be a flavanoid (e.g. flavan3ol).

Most preferred combination therapies comprise a rocket extract rich in ITCs and a grape extract rich in polyphenols.

Preferred plant extracts comprising polyphenols comprise procyanadins and are derived from Grape skin and/or Grape seeds. Powdered grape skin/seed extract may be made using methods known to the art. Such powders may be further processed before being used according to the invention. For instance powdered grape skin/seed extract may be dissolved in MeOH; heated to about 70° C. (e.g. for 10-30 minutes); centrifuged (e.g. at about 4500 rpm for 15 minutes); and filtered to 0.45μ. This provides a solution comprising procyanidin which may be concentrated, powdered or diluted (as required) and used according to the invention.

Most Preferred plant extracts comprising polyphenols are prepared using fed or white grape skins (ideally with their seeds and stalks) as a starting material for extraction. Vinification process solid wastes represent an ideal starting material. Fresh grapes, preferably seeded, are another suitable starting material. The most ideal starting material contains proportionally more seeds and stalks than skins.

If obtained fresh, for example vinification solid waste, the grapes skin/seed mixtures can be dried by air drying, for example on a heated belt dryer. The dried starting material should then be milled finely to produce a powder with particle size <250 micron. Preparation of high-polyphenol extracts, containing a high proportion of procyanidins, can be carried out by continuous extraction, preferably by counter-current extractors, in either ethanol/water mixtures, or acetone/ethanol/water mixtures. The extractants may be acidified by addition of, for example, hydrochloric, citric or tartaric acids, so that the pH range is between 1.5 and 4, to improve recovery if a high proportion of grape skins is present. This is not always necessary, especially if the proportion of seeds is high. The extractants should contain between 45% and 65% ethanol, and may contain in addition up to 15% acetone. Extraction may be carried out in a single pass, but preferably two or three sequential extraction stages may be employed to maximise recovery. Once extraction is complete, solids can be removed from the suspension by centrifugal separation or decanting. The procyanidin-rich supernatant can be deproteinated by chemical or enzymatic means, or by filtration (e.g. ultrafiltration), and concentrated by low-temperature high vacuum evaporation, or by removal of water by reverse osmosis. The final extract can be stored frozen as a liquid or spray-dried to give a powder, or encapsulated (e.g. in a fat matrix, or in a polysaccharide matrix, or in a polymer matrix) to enhance stability.

Preferred extracts comprising polyphenols at a concentration of procyanidins of between 0.1 and 10 g/L and preferably between 0.5 and 1.5 g/L.

A preferred composition according to the seventh aspect of the invention comprises a grape juice rich in a polyphenol such as procyanadin and a rocket extract rich in SF or ER. The inventors have found that such compositions are particularly effective for preventing or reducing inflammatory reactions.

Alternatively liquid formulations and powders comprising polyphenols may be exploited according to the seventh aspect of the invention.

Most preferred compositions according to the seventh aspect of the invention comprise a therapeutically effective amount of procyanadins derived from grape skin or grape seeds and a therapeutically effective amount of an ITC (e.g. derived from rocket). Such compositions may comprise a foodstuff or drink comprising powders containing the procyanadin and ITC. However most preferred compositions comprise encapsulated liquids, semisolids or powders (as contemplated above) containing a procyanadin and a ITC. It is most preferred that the composition is a gelatine encapsulated liquid comprising a concentration of ITCs between 1 and 1000 μM and preferably between 10 and 100 μM and a concentration of procyanidins of between 0.1 and 10 g/L and preferably between 0.5 and 1.5 g/L.

Encapsulation of the ITC-rich extract/procyanidin-rich extract may be undertaken in order to a) enhance stability of the extract by preventing exposure to oxidation and b) alter the sensory characteristics of the extracts/mixtures (e.g. to reduce odour). Encapsulation can be carried out by first preparing a solution of the extracts in ethanolic solution at a concentration of between 50% and 70% dry matter. The concentration of ethanol may be between 0% and 10%. The proportion of ITC extract to procyanidin extract may be 3:1 or 5:1 or 10:1. The prepared solution should be mixed in equal volumes with a suitable encapsulant shell matrix. For example, a mixture of fats, or a solution of polysaccharides such as alginates, or a solution of polymeric material such as chitosan. The mixture should be thoroughly homogenised at a temperature not exceeding 90° C., and formed into particles by either spray drying, or by forming an aerosol and cooling, or by other known encapsulation techniques. Final particle size should not exceed 100 micron. The resulting encapsulates may be either hard-shell or soft-shell, and should contain a minimum of 10% extract w/w, but preferably between 20% and 50% extract w/w.

### Dose Regimens

The compositions of the invention can be presented in the form of unit dosage forms containing:

(a) a defined concentration of ITC (or precursors thereof) or plant extract comprising ITC or precursors thereof, as defined by the first-sixth aspects of the invention; or

(b) a defined concentration of ITC (or precursors thereof) or plant extract comprising ITC or precursors thereof and a defined concentration of a polyphenol or a plant extract comprising a polyphenol as defined by the seventh aspect of the invention.

Such unit dosage forms can be selected so as to achieve a desired level of biological activity.

The amount of a composition according to the invention required by a subject is determined by biological activity and bioavailability which in turn depends on the formulation, mode of administration, the physicochemical properties of the ITC or plant extract and whether the ITC or extract is being used as a monotherapy or in a combined therapy (e.g. with a polyphenol according to the seventh aspect of the invention). Generally, a daily dose for a human adult should be between 0.1 g and 100 g of freeze-dried or spray-dried powder (however formulated), more preferably the daily dose is between 1 g and 30 g (e.g. about 5 g, 10 g, or 15 g as required).

A solid or semisolid dosage form of the present invention can contain up to about 1000 mg of dried extract containing ITC or precursor thereof.

The frequency of administration will also be influenced by the above-mentioned factors and particularly the half-life of the ITCs, or precursors thereof, and the half-life of the polyphenol (if used) within the subject being treated. For instance, the half-life will be influenced by the health status of the subject, gut motility and other factors.

The compositions according to the invention may be included in a pharmaceutical formulation such as a tablet or a capsule. Such formulations may be required to be enterally-coated if bioavailability dictates this. Known procedures, such as those conventionally employed by the pharmaceutical industry (e.g. in vivo experimentation, clinical trials etc), may be used to establish specific formulations of pharmaceutical compositions and precise therapeutic regimes (such as daily doses and the frequency of administration).

It will be appreciated that conventional nutraceutical procedures may be employed to create liquid drinks, powder mixes and food-stuffs comprising the compositions.

Daily doses may be given as a single administration (e.g. a daily tablet for oral consumption or as a single liquid drink). Alternatively administration may be required twice or more times during a day. As an example, a 100 ml orange or grape drink containing 0.1-20 g of spray dried plant extract (preferably 0.3-10 g of spray dried rocket extract and more preferably 0.5-3.0 g) may be used to quench thirst at regular intervals throughout the day and thereby deliver a recommended dose. It will be appreciated that the combination of grape and rocket extract will represent a most preferred composition according to the seventh aspect of the invention.

It will be appreciated that nutritional products supplemented with ITC (and/or polyphenols) or plant extracts according to the invention represent an ideal means for providing subjects with, or at risk of developing, medical conditions with inflammatory components with a protective or therapeutically effective amount of ITC. Therefore, according to an eighth aspect of the present invention there is provided a nutritional product for use in the prevention or treatment of medical conditions characterised by having an inflammatory component wherein the product is supplemented with ITC or a precursor thereof; or a plant extract enrich with ITC or a precursor thereof.

The nutritional product may comprise:
(a) a clear, low viscosity, water-like, stable, ready-to-use, bottled, carbonated or non-carbonated drink; or a concentrated clear liquid for reconstitution containing a plant extract according to the fourth aspect of the invention;
(b) a powder/granular mix to be reconstituted with water or any other orally ingestible liquid as a drinkable liquid, containing a plant extract according to the fourth aspect of the invention; or
(c) a powder/granular mix mixed into a food stuff (e.g. a chocolate bar, lozenge or the like).

The nutritional product may be as described above and may or may not contain water-soluble vitamins, additional mineral supplements, nutritional compounds, antioxidants or flavourings.

Preferred nutritional products may comprise the active ingredients defined by the seventh aspect of the invention.

The present invention will be further illustrated, by way of examples, with reference to the accompanying drawings in which:

FIG. 1 is a schematic illustrating the metabolism of 4-methylsulphinylbutyl glucosinolate and sulforaphane. Upon entry into enterocytes sulforaphane (SF) is rapidly conjugated to glutathione, exported into the systemic circulation and metabolized through the mercapturic acid pathway. Within the low glutathione environment of the plasma the SF-glutathione conjugate may be cleaved, possibly mediated by GSTM1, leading to circulation of free SF in the plasma. This free SF can modify plasma proteins including signalling molecules, such as TGFβ, EGF and insulin.

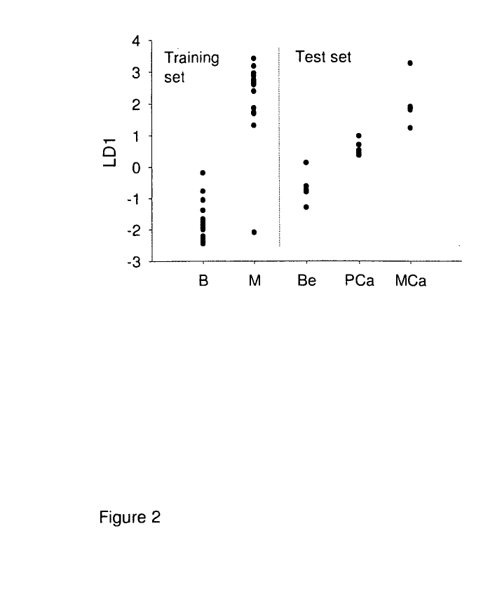

FIG. 2 is a plot showing Linear discriminant analysis (LDA) of an independent prostate microarray data set using the benign (B) and malignant (M) TURP prostate tissue referred to in Example 1 as training samples to classify the laser-capture microdissected (LCD) epithelial prostate cell samples (GEO Accession: GDS1439), consisting of benign (Be), primary cancer (PCa) and metastatic cancer (MCa) samples. LDA was performed on a gene list that distinguished the benign and malignant TURP samples as described in the methods section of Example 1. Here, the first linear discriminant (LD1) is shown.

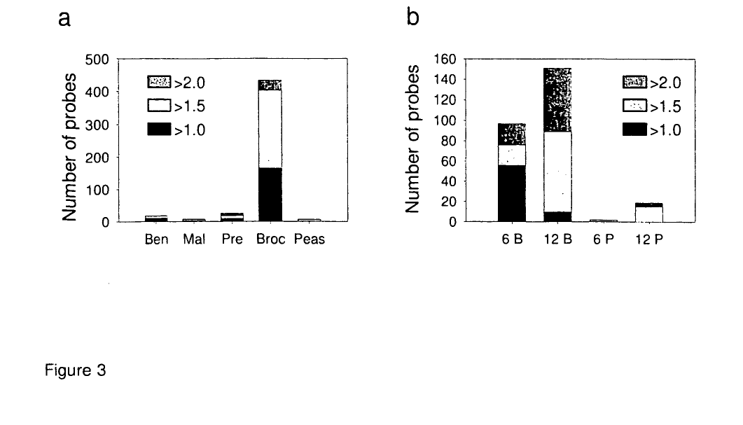

FIG. 3 graphically represents the effect of dietary intervention on gene transcription as described in Example 1. a, Number of probes that differ between GSTM1 positive and null genotypes (P≦0.005, Welch modified two-sample t-test) in TURP tissue from benign (Ben) and malignant (Mal) prostates, and TRUS-guided biopsy tissue from volunteers at pre-intervention (Pre), post 6 months broccoli-rich diet (Broc) and post 6 months pea-rich diet (Peas). b, Number of probes that differ between pre-intervention TRUS-guided biopsy samples and after 6 months broccoli (6B)-, 6 month pea (6P)-, 12 month broccoli (12B)- and 12 month pea (12 P)-rich diets (P≦0.005, Welch modified two-sample paired t-test). Shading correspond to different fold cutoffs applied as discussed in Example 1.

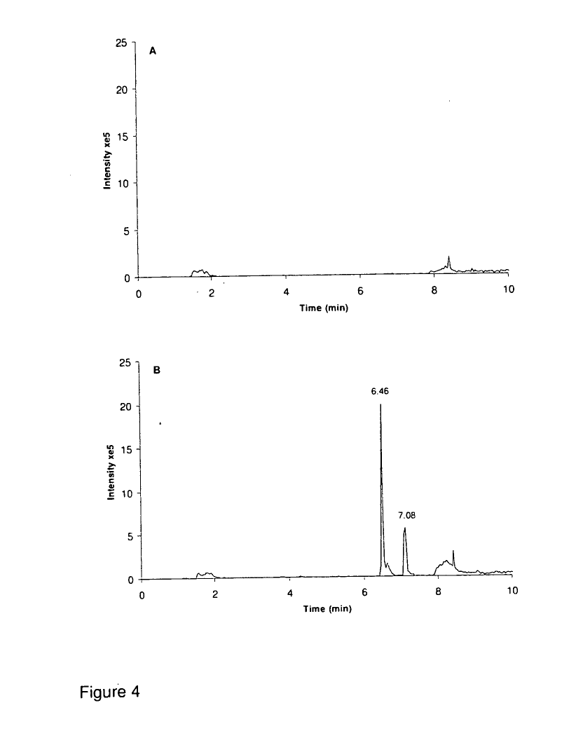

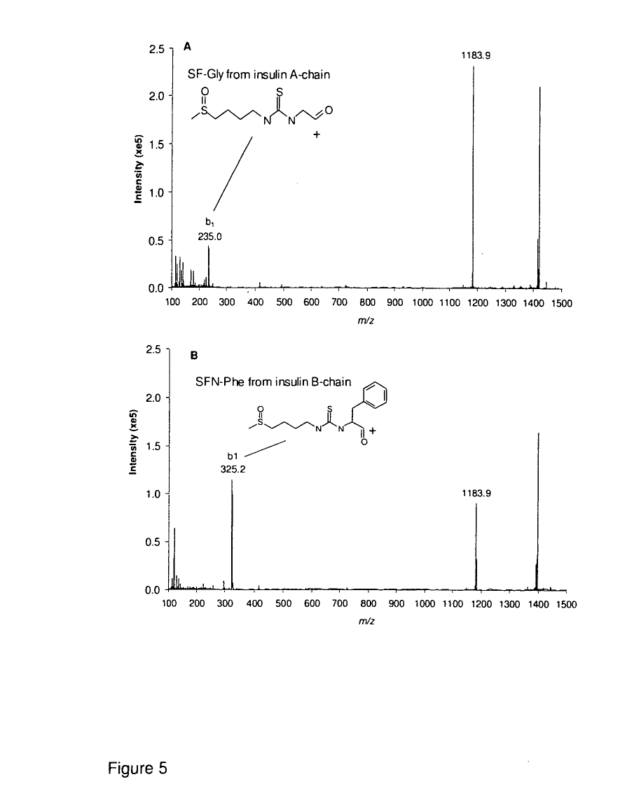

FIG. 4 represents LC-MS traces of insulin incubated with and without SF in human plasma as discussed in Example 1. Extracted ion LC-MS chromatograms (m/z 1183.6-1184.1) of insulin-SF MH₅⁵⁺ in (A) unmodified insulin (20 μg/ml) in human plasma control and (B) human plasma incubated with insulin (20 μg/ml) and 50 μM SF for 4 h at 37° C., showing the appearance of two different insulin-SF conjugates at retention times of 6.46 and 7.08 min. The enhanced product ion (EPI)-MS spectra of these two insulin-SF conjugates are shown in FIG. 5.

FIG. 5 represents enhanced product ion (EPI)-MS spectra of the two insulin-SF conjugates as discussed in Example 1. MS² product ion spectra of (A) 6.46 min and (B) 7.08 min retention time peaks from LC-MS analysis of human plasma incubated with bovine insulin and 50 μM SF for 4 h at 37° C. In (A) and (B) m/z 1183.9 corresponds to insulin-SF MH₅⁵⁺ and in (A) m/z 235.0 corresponds to Gly-SF, the N-terminal amino acid of insulin A chain and in (B) m/z 325.2 corresponds to Phe-SF, the N-terminal amino acid of insulin B chain.

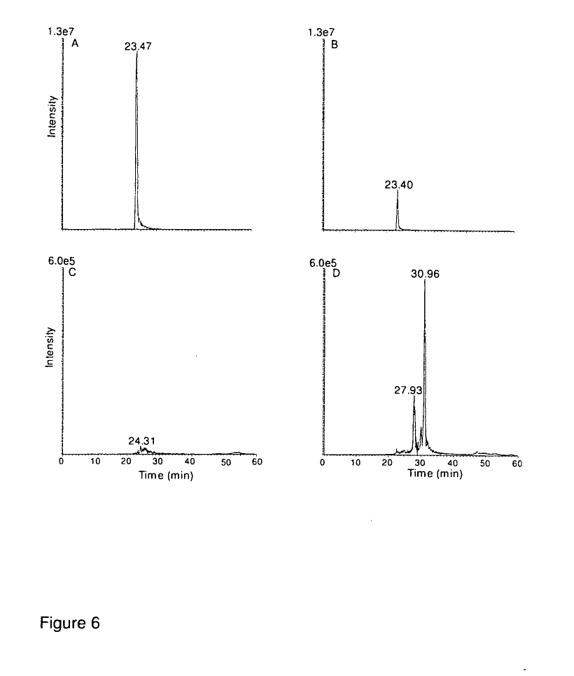

FIG. 6 illustrates LC-MS of TGFβ1 incubated with and without SF as discussed in Example 1. Extracted ion chromatograms (MS) of precursor masses representing the unmodified N-terminal peptide of TGFβ1 (m/z 768.5) and the modified N-terminal peptide (m/z 877.2) A of m/z 768.2-769.2 from DMSO treated TGFβ1, B of m/z 768.2-769.2 from SF treated TGFβ1, C of m/z 876.7-877.7 DMSO treated TGFβ1 and D of m/z 876.7-877.7 SF treated TGFβ1.

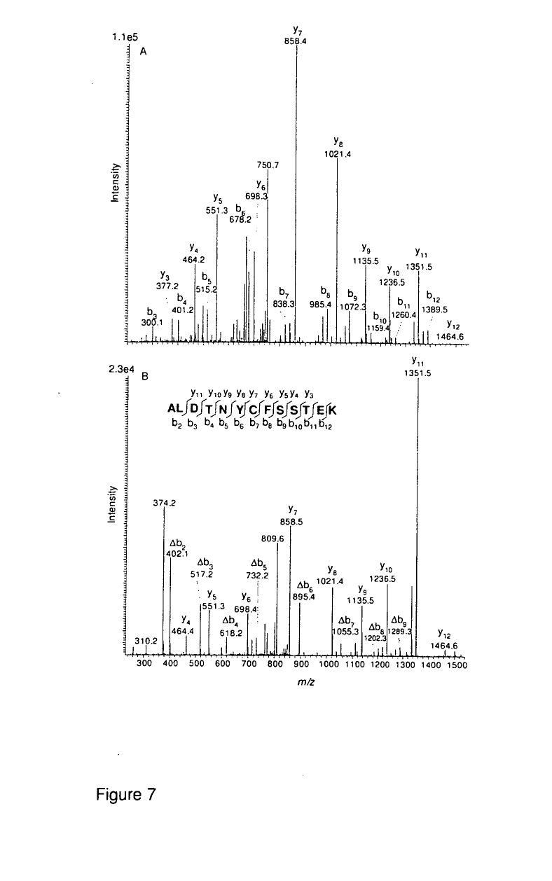

FIG. 7 illustrates N-terminal modification of TGFβ1 by SF. MS/MS spectra of m/z 768.7 representing the unmodified N-terminal peptide of TGFβ1 at retention time 23.43 min (A) and m/z 877.2 representing a modified form of TGFβ1 seen only in SF treated samples at retention time 30.85 minutes (B). Note that the y ion series remains the same while the b ion series shifts (Δ) indicating an N-terminal modification of mass 217±0.8 Da. FIG. S2 provides an explanation of the mass addition of 217, as opposed to 177.

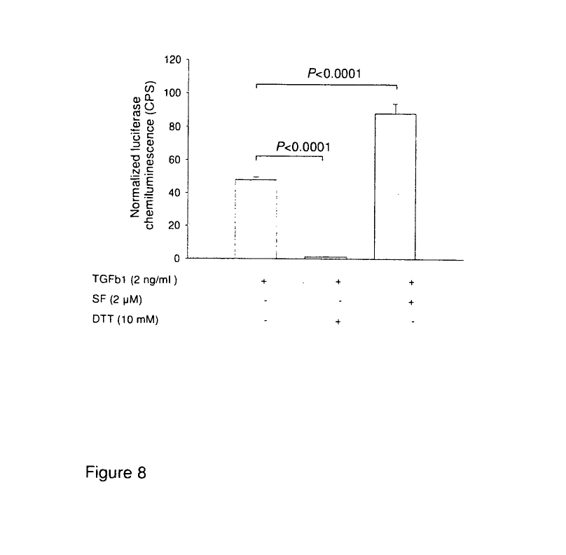

FIG. 8 illustrates activation of TGFβ1/Smad mediated transcription by SF as discussed in Example 1. NIH3T3 cells containing a CAGA12-luc plasmid were treated with TGFβ1 alone, TGFβ1 and 10 mM DTT, which disrupts the active TGFβ1 dimer, or TGFβ1 and 2 μM SF. All samples were pre-incubated for 30 minutes and further dialyzed for 4 h so that the final concentration of SF was 34 nM. As an additional negative control cells received no treatment or only 34 nM SF, both of which failed to induce luciferase. Chemiluminescence was normalized to the protein concentration of each sample (for details see Methods). This is a representative experiment of a total of four similar experiments performed. Data shown are mean (s.e.m) of three replicates.

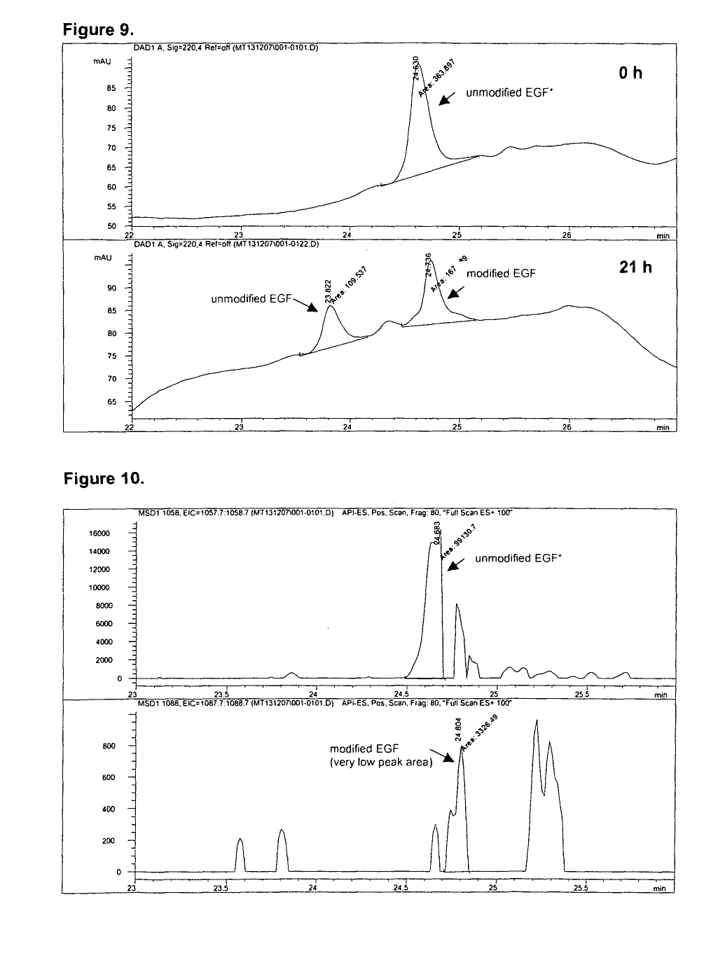

FIG. 9 represents a UV spectrum of EGF at 220 nm wavelength after 0 h and 21 h incubation with SF as discussed in Example 2 (Expt 1). * The unmodified EGF runs later (at 24.630 sec) at the 0 h sample compared to the 21 h sample (23.822 sec) because of column equilibration.

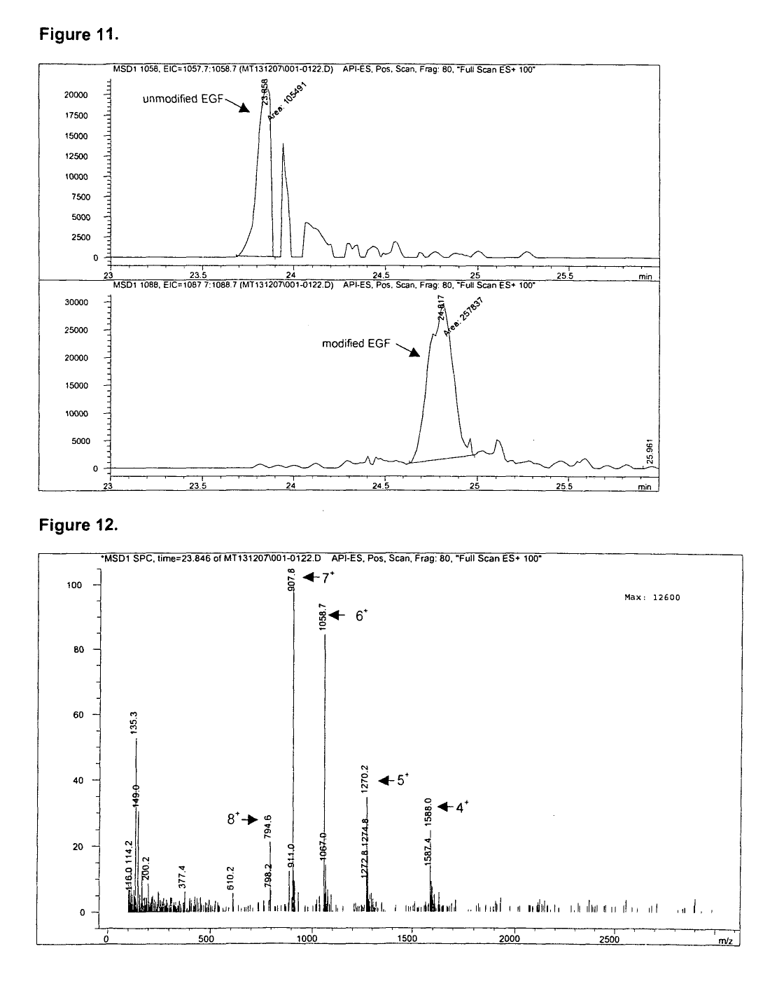

FIG. 10 represents mass spectrum of extracted ions for the unmodified and modified EGF at 0 h as discussed in Example 2 (Expt 1). * The unmodified EGF runs later at the 0 h sample (FIG. 10 top panel) compared to the 21 h sample (FIG. 11 top panel) because of column equilibration.

FIG. 11 represents mass spectrum of extracted ions for the unmodified and modified EGF at 21 h as discussed in Example 2 (Expt 1).

FIG. 12 represents mass spectrum of the unmodified EGF as discussed in Example 2 (Expt 1). Shown are multiple charged EGF molecules.

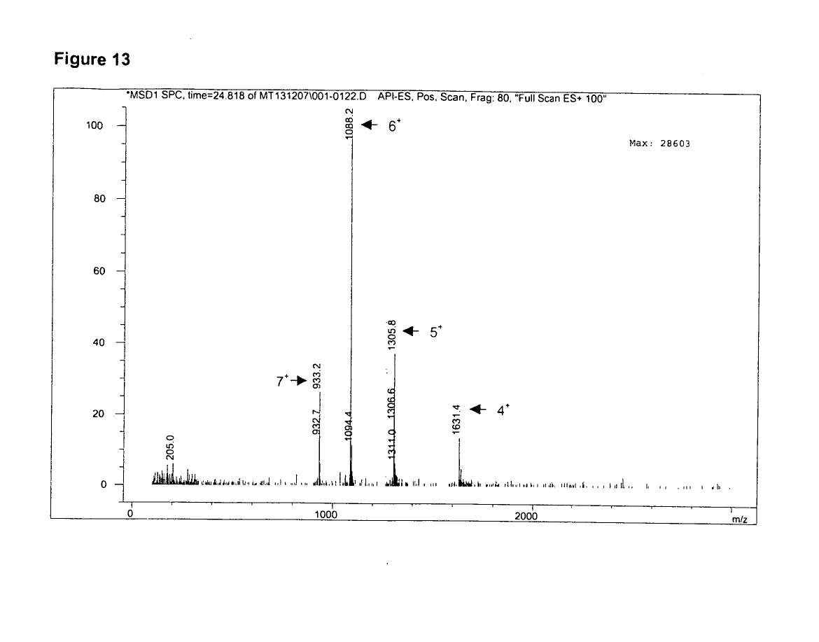

FIG. 13 represents a mass spectrum of the modified EGF. Shown are multiple charged modified-EGF molecules as discussed in Example 2 (Expt 1).

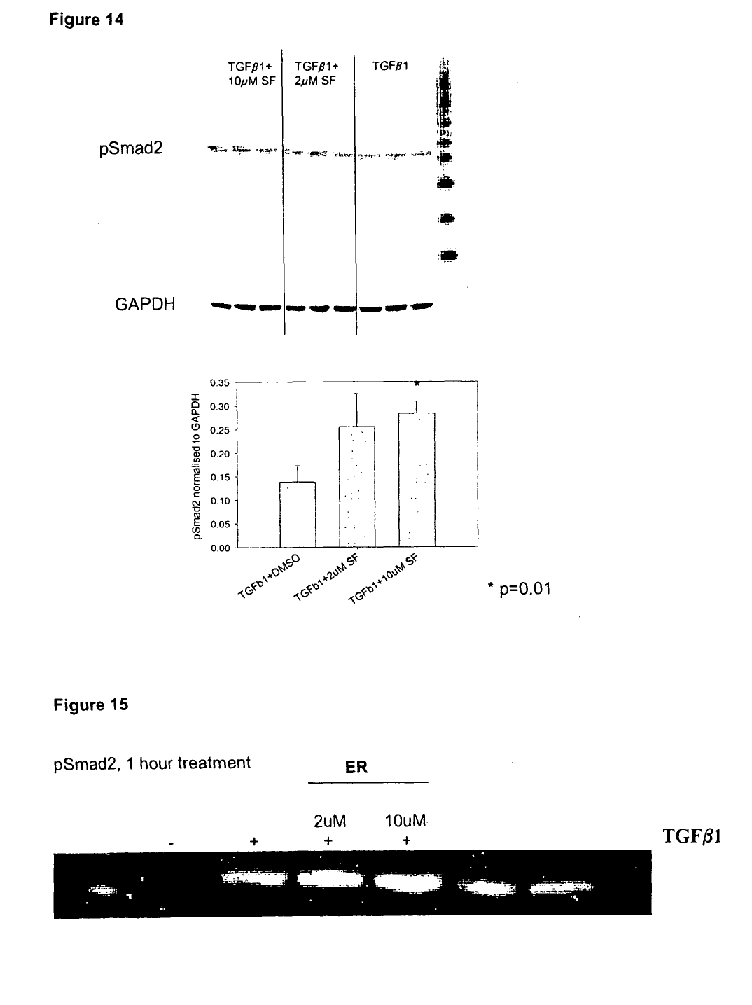

FIG. 14 represents (a) a photograph of a gel; and (b) the data quantified in a bar chart which corresponds to the data presented in Table 6 as discussed in Example 2 (Expt 2). The data illustrates how an ITC modulates Smad activity. The experiment concerned pre-incubation of TGFβ1 with and without sulforaphane (SF) for 30 minutes before treating PC3 cells for 1 h and then measuring Smad2 phosphorylation (i.e. a function of TGFβ1 activity).

FIG. 15 represents a photograph of a gel; and corresponds to the data which was quantified and presented in Table 7 as discussed in Example 2 (Expt 2). The data illustrates how an ITC modulates Smad activity. The experiment concerned pre-incubation of TGFβ1 with and without Erucin (ER) for 30 minutes before treating PC3 cells for 1 h and then measuring Smad2 phosphorylation (i.e. a function of TGFβ1 activity).

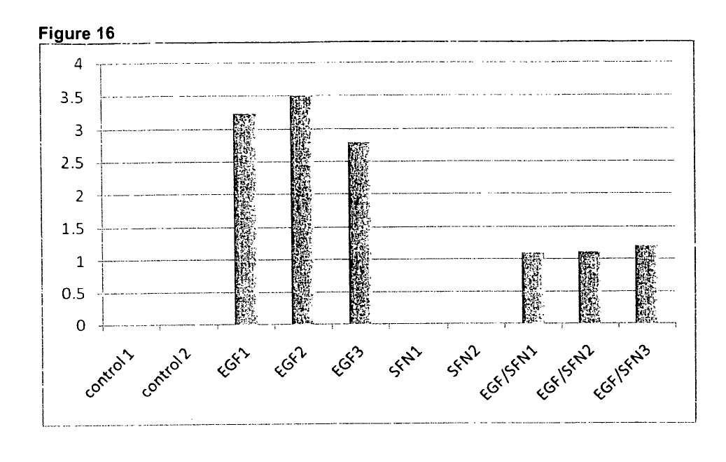

FIG. 16 represents a bar chart showing Expression of phosphorylated EGF receptor (p-EGFR) in BPH1 cells (hyperplastic prostate cells) as discussed in Example 2 (Expt 3). The experiments involved pre-incubation of BPH cells with 10 μmol/L sulforaphane results in approximately threefold reduction in EGF receptor phosphorylation inducible by 10 mg/L EGF over a 10 minute timecourse.

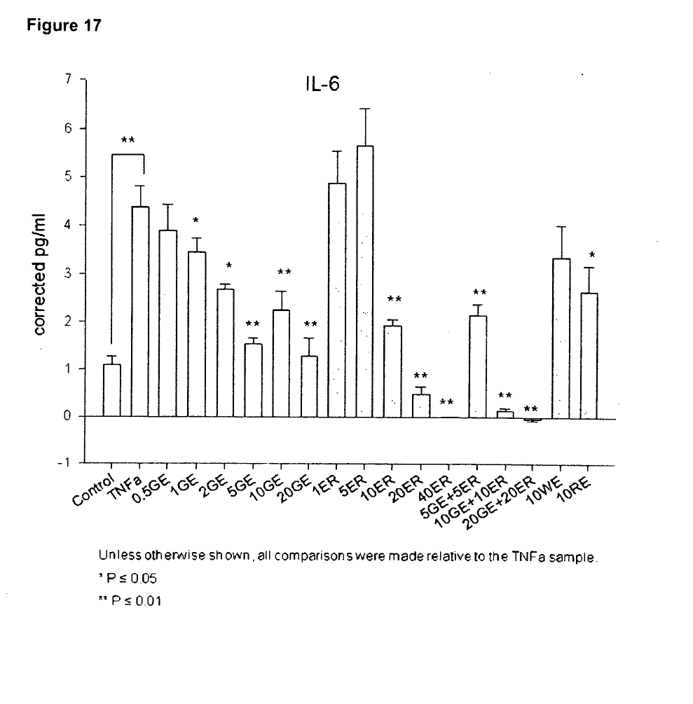

FIG. 17 represents a bar chart illustrating the effect of incubation of HUVEC cells with high procyanidin extracts and with erucin on IL-6 expression as discussed in Example 3.

### EXAMPLE 1

The present invention is based on work, conducted by the inventors, that investigated the effect of consuming cruciferous vegetables on the risk of both the incidence of prostate cancer and of developing aggressive prostate cancer and in particular the underlying mechanisms of action that lead to such a cancer. In this study, the inventors quantified and then interpreted changes in global gene expression patterns in the human prostate gland before, during and after a 12 month broccoli-rich diet. The results made the inventors realise that ITCs, present in the vegetables, modulated signal transduction mechanisms that controlled the inflammatory reaction as-well-as modulating the progression of prostate cancer. This realisation lead the inventors to develop the compositions and plant extracts contemplated herein and their uses in treating conditions with an inflammatory component.

Volunteers were randomly assigned to either a broccoli-rich or a pea-rich diet. After six months there were no differences in gene expression between glutathione S-transferase mu 1 (GSTM1) positive and null individuals on the pea-rich diet but significant differences between GSTM1 genotypes on the broccoli-rich diet, associated with transforming growth factor beta 1 (TGFβ1) and epidermal growth factor (EGF) signalling pathways. Comparison of biopsies obtained pre and post intervention revealed more changes in gene expression occurred in individuals on a broccoli-rich diet than in those on a pea-rich diet. While there were changes in androgen signalling, regardless of diet, men on the broccoli diet had additional changes to mRNA processing, and TGFβ1, EGF and insulin signalling. The inventors also established that sulforaphane (SF: the isothiocyanate derived from 4-methylsuphinylbutyl glucosinolate that accumulates in broccoli) chemically interacts with TGFβ1, EGF and insulin peptides to form thioureas, and enhances TGFβ1/Smad-mediated transcription.

Prostate cancer is the most frequently diagnosed non-cutaneous cancer within the male population of western countries. Epidemiological studies have suggested that diets rich in cruciferous vegetables, such as broccoli, may reduce the risk of prostate cancer, in addition to cancers at other sites and myocardial infarction. Some studies have specifically demonstrated that consuming one or more portions of broccoli per week can reduce the incidence of prostate cancer, and also the progression from localized to aggressive forms of prostate cancer. The reduction in risk may be modulated by glutathione S-transferase mu 1 (GSTM1) genotype, with individuals who possess at least one GSTM1 allele (i.e. approximately 50% of the population) gaining more benefit than those who have a homozygous deletion of GSTM1. One object of this study was therefore to investigate the mechanistic basis to the protective effect of broccoli and the interaction with GSTM1 genotype

These findings made the inventors realise that consuming broccoli interacts with GSTM1 genotype to result in complex changes to signalling pathways associated with inflammation as-well-as carcinogenesis in the prostate. The inventors believe that these changes may be mediated through the chemical interaction of ITCs with signalling peptides in the plasma. This study provides, for the first time, experimental evidence obtained in humans to support observational studies that diets rich in cruciferous vegetables may reduce the risk of developing and/or treat conditions with an inflammatory component. Furthermore the inventors went on to establish (see subsequent examples) that products comprising ITCs or comprising plant extracts enriched in ITCs will be useful for treating such conditions.

### 1.1 Methods

### 1.1.1 Subjects and Study Design

Twenty two men aged 57-70 years (Table 1) with a previous diagnosis of high-grade prostatic intraepithelial neoplasia (HGPIN), the pre-invasive in situ stage of prostatic adenocarcinoma, were recruited into a dietary intervention trial to study the effects of a diet rich in broccoli and a diet rich in peas on prostate gene expression.

Histological diagnosis was made by two consultant histopathologists, who had a special interest in prostate pathology. Ethical approval for the trial was obtained and all participants gave written, informed consent. Volunteers were excluded if they were undergoing chemopreventive therapy, were receiving testosterone replacement medication or 5 alpha reductase inhibitor, had active infection requiring treatment, had a body mass index (BMI)<18.5 or >35, or were diabetic. Subjects were allocated into a 12-month, parallel dietary intervention trial consisting of two dietary intervention groups: (i) consuming 400 g broccoli per week or (ii) consuming 400 g peas per week, in addition to their normal diet. The trial was conducted from April 2005-April 2007. Plasma prostate specific antigen (PSA) levels were quantified prior to the intervention study and after six and 12 months at the Norfolk and Norwich University Hospital with the use of a total PSA immunoassay. Volunteers avoided foods known to contain glucosinolates for 48 hours prior to each biopsy appointment to avoid acute effects.

In addition to the transrectal ultrasound scan (TRUS)-guided needle biopsies of the prostate obtained from the volunteers immediately prior to the intervention study, and after six and twelve months, 18 benign and 14 malignant transurethral resection of the prostate (TURP) tissues were also obtained from the Norfolk & Norwich University Hospital Partners in Cancer Research Human Tissue Bank.

### 1.1.2 Dietary Intervention

Vegetables were delivered to the volunteers on a monthly basis. They were provided with a steamer and the volunteers were given a demonstration by the diet cooks at the Institute of Food Research of how to cook the vegetables. Portions of broccoli were steamed for 4-5 minutes and portions of peas were steamed for 2-3 minutes. Frozen peas (Birds Eye Garden Peas, http://www.birdseye.co.uk/) were purchased from a local retail outlet. To ensure consistency in glucosinolate content in frozen broccoli provided to the volunteers, the broccoli required for the intervention study was grown in one batch at an ADAS experimental farm at Terrington, near King's Lynn, UK (http://www.adas.co.uk/) and processed by Christian Salvesen (Bourne, Lincolnshire, UK, http://www.salvesen.co.uk/). It was blanched at 90.1° C. for 74 s, frozen at −30° C. and packaged into 100 g portions, then stored at −18° C. until steamed by the volunteer. The broccoli was a high glucosinolate variety. The levels, mean (SD), of 4-methylsulphinylbutyl and 3-methylsulphinylpropyl glucosinolates (the precursors of SF and IB, respectively) were 10.6 (0.38) and 3.6 (0.14) μmolesg⁻¹ dry weight, respectively, compared to 4.4 (0.12) and 0.6 (0.01) μmolesg⁻¹ dry weight in broccoli purchased from local retail outlets. Although the level of glucosinolates were higher than standard broccoli, blanching prior to freezing denatured plant myrosinase, thus the levels of SF and IB derived from the high glucosinolate broccoli diet would be similar to or lower than those obtained from fresh broccoli with functional myrosinase. Levels of indole glucosinolates were similar in both high glucosinolate and standard broccoli.

### 1.1.3 Compliance Monitoring and Dietary Assessment

Volunteers completed weekly tick sheets during the 12-month intervention period to identify when the portions of vegetables were eaten. Every two weeks, volunteers were contacted by telephone and asked about adherence to the diet. A seven-day estimated food intake diet diary was completed by volunteers at baseline and after six months using household measures as an indication of portion size. Food intake from the diaries was inputted into Diet Cruncher v1.6.1 (www.waydownsouthsoftware.com/) and analyzed for differences in nutrient composition between the two intervention groups at baseline and six months after intervention.

### 1.1.4 Genotyping

Genomic DNA was extracted from whole blood or from tissue samples using Qiagen QIAamp DNA minikit with RNase treatment according to the manufacturer's instructions (http://www.qiagen.co.uk/). GSTM1 (NM_{—}000561) genotype was determined using a real-time PCR procedure based on Covault and colleagues, using gene specific primers and probe and quantified relative to a two-copy gene control, a region in IVS10 of the breast cancer 1, early onset (BRCA1, NM_{—}007294) gene (Covault et al. (2003) Biotechniques 35: 594-596, 598). Primers and probes were designed using Applied Biosystems Primer Express (http://www.appliedbiosystems.com/) and are given with PCR conditions in Table 1. Data were analyzed with Applied Biosystems Absolute Quantification software.

In table 1: Sequences and concentration of forward (F) and reverse (R) primers and fluorogenic probes (P) for the determination of GSTM1 gene deletion are shown. Probes were labelled with a 5′ reporter dye, FAM (6-carboxyfluorescein) and a 3′ quencher dye, TAMRA (6-carboxytetramethylthodamine). Triplicate reactions were carried out in a total volume of 25 μL/well consisting of Universal MasterMix, primers and probes and 50 ng DNA. Amplitaq Gold activation for 10 min at 95° C., followed by 40 cycles PCR of denaturation for 15 s at 95° C. and annealing/extension for 1 min at 60° C.

TABLE 1
Primers and probes for genotype analysis

| | | |
|---|---|---|
| | | primer |
| | | & |
| | | probe |
| Gene | Sequence | (nM) |

GSTM1-glutathione S-transferase M1

| | | |
|---|---|---|
| F | 5′-GGAGACAGAAGAGGAGAAGATTCG-3′ | 500 |
| | (SEQ ID No. 1) | |
| R | 5′-TGCCCAGCTGCATATGGTT-3′ | 500 |
| | (SEQ ID No. 2) | |
| P | 5′-TCCATGGTCTGGTTCTCCAAAATGTCCA-3′ | 200 |
| | (SEQ ID No. 3) | |

Control gene (BRCAl)

| | | |
|---|---|---|
| F | 5′-GTCTGCTTTTACATCTGAACCTCTGT-3′ | 500 |
| | (SEQ ID No. 4) | |
| R | 5′-AGCCCTGAGCAGTCTTCAGAGA-3′ | 500 |
| | (SEQ ID No. 5) | |
| P | 5′-ACTCTCACACCCAGATGCTGCTTCACCT-3′ | 200 |
| | (SEQ ID No. 6) | |

One of the 22 volunteers was diagnosed with prostatic adenocarcinoma at the study baseline biopsy and was removed from the study. Eleven samples from the baseline biopsies, two samples from the six-month biopsies and three samples from the 12-month biopsies did not produce good quality RNA and/or sufficient cRNA and were not hybridized. In addition, one volunteer showed prostatic adenocarcinoma at the six-month biopsy; subsequent samples were removed from the study. Fluorescence intensity for each array was captured with a GeneChip ® Scanner 3000 7G. Affymetrix GeneChip ® Operating Software (GCOS) was used to quantitate each U133 Plus 2.0 array. Microarray data in this paper are compliant to the minimum information about a microarray experiment (MIAME) criteria and are deposited at Array Express (http://www.ebi.ac.uk/microarray-as/aer; Accession Number E-MEXP-1243).

### 1.1.5 RNA Extraction and Microarray Hybridisation

Total RNA was isolated from the TURP tissue bank samples and the TRUS-guided needle biopsies from the volunteers with the use of QIAGEN® RNeasy mini kits according to the manufacturer's instructions (http://www.qiagen.co.uk/). The quantity of resulting RNA was measured using a spectrophotometer (Beckman). The RNA quality was determined using the Agilent 2100 Bioanalyzer (http://www.agilent.co.uk/). RNA samples from TURP biopsies of benign and malignant prostates and from TRUS-guided biopsies from both subject groups (peas and broccoli) at baseline, and at six and 12 months after intervention were hybridized onto Affymetrix Human U133 Plus 2.0 microarrays (http://www.affymetrix.com/) by the Nottingham Arabidopsis Stock Centre (NASC, http://arabidopsis.infof). Double-stranded cDNA synthesis and generation of biotin-labeled cRNA were performed according to the manufacturer's protocol (Affymetrix, http://www.affymetrix.com/). The final cRNA was checked for quality before fragmentation and hybridization onto the arrays.

### 1.1.6 Microarray Data Analysis

Raw data files (CEL) were loaded into the DNA-Chip Analyzer software (dChip, http://biosun1.harvard.edu/complab/dchip/, build date September 2006) for normalization, generation of expression values and statistical analysis. Following normalization using the Invariant Set Normalization method, probe expression levels were calculated using the PM-only model. To identify genes that were changing between groups, different two-tailed P-value thresholds were applied calculated by Welch modified two-sample t-test in dChip. Paired or unpaired t-tests were performed as appropriate. To correct for multiple testing, False Discovery Rate (FDR) was estimated by permutation in dChip and the median of 100 permutations reported for each of the comparisons (1000 permutations on selected samples had little effect on FDR calculations). Unsupervised clustering was performed on benign and malignant samples using 1-Rank correlation as distance metric on a gene list of 3697 probes. These probes satisfied two criteria: first, that the coefficient of variation (CV) was between 0.5 and 1000; and secondly, that the percentage of Presence calls was more than 20% across all TURP benign and malignant samples.

For the purpose of sample classification, 19 laser-capture microdissected (LCD) epithelial cell microarrays (GEO Accession: GDS1439, http://www.ncbi.nlm.nih.gov/geo/) and 32 TURP benign and malignant microarrays were normalized together and model-based expression was calculated as described above in dChip. The LCD samples were derived from six benign prostate tissue samples, five clinically localized primary prostatic adenocarcinoma samples, two replicates of the five primary cancer samples after pooling, four metastatic prostatic adenocarcinoma samples and two replicates of the four metastatic prostate cancer samples after pooling (Varambally et al. (2005) Cancer Cell 8: 393-406). Classification of the LCD epithelial cell samples was then performed using linear discriminant analysis (LDA) based on the TURP benign and malignant samples as training samples. LDA was performed using 442 probes that had higher than 100 units difference in signal intensity between TURP benign and malignant samples and were significantly different at P≦0.01 by Welch modified two-sample t-test. To identify pathways that are the most over-presented in the lists of differentially expressed genes, functional analyses using MAPPFinder and GenMAPP v2.1 were performed (http://www.genmapp.org/).

1.1.7 Incubation of Peptides with Isothiocyanates

Incubations of SF or IB with bovine insulin (P01308, Sigma-Aldrich), recombinant human epidermal growth factor (EGF, P01133, R&D Systems, http://www.rndsystems.com/) and recombinant human transforming growth factor beta 1 (TGFβ1, P01137, R&D Systems) were performed in sodium phosphate-buffered saline solution (pH 7.4) or human blood plasma at 37° C. for 0.5-24 h. Plasma was pre-treated by ultrafiltration to remove high molecular weight proteins (Microcon Ultracel YM-30 filter, MWCO 30,000). Samples were either analyzed directly by LC-MS/MS or by LC-MS/MS analysis of tryptic digests of gel electrophoresis bands.

1.1.8 Direct LC-MS/MS Analysis of Peptides Incubated with Isothiocyanates

The LC system used was a Shimadzu series 10AD VP (Shimadzu, http://www.shimadzu.com/). The column was an ACE 300 C18, 150×2.1 mm (5 μm particle size) used at 40° C. Mobile phase A was 0.1% formic acid in water, mobile phase B, 0.1% formic acid in acetonitrile and the flow rate was 0.25 ml/min. A linear gradient was used from 25% B to 35% B over 0 to 5 min, then a further gradient from 35% B to 99% B over 6 min followed by 99% B for 4 min. The column was re-equilibrated for a total of 3 min. The injection volume was between 5-20 μl. All MS experiments were conducted on a 4000 QTRAP hybrid triple-quadrupole linear ion trap mass spectrometer using Analyst version 1.4.1 software (Applied Biosystems, http://www.appliedbiosystems.com/) equipped with a TurboIon source used in positive ion electrospray mode. The probe capillary voltage was optimized at 4200 V, desolvation temperature set to 400° C., curtain gas, nebulizing and turbo spray gas were set to 40, 10 and 20, respectively (arbitrary values). Declustering potential was ramped between 50-120 V. Nitrogen was used for collisionally induced dissociation (CID). The peak-width was set on Q1 and Q3 at 1.0 Th (measured at half height) for all MS and MS/MS experiments. Spectra were obtained over the range m/z 800-2000 with scan times of 1-2 sec. Operating in LIT mode Q0 trapping was activated and dynamic fill time used, the scan rate was set to 250 Th/s for enhanced product ion (EPI) scans, excitation time was 150 msec, excitation energy 25 V and entry barrier 4 V. For EPI spectrum acquisition the precursor ions of interest for conjugates of SF with insulin (m/z 1183.9 MH₅⁵⁺), EGF (m/z 1088.8, MH₅⁶⁺) and TGFβ (m/z 1981.9, MH₅¹³⁺) were selected, the collision energy was ramped between 30-120V and spectra were obtained over the range m/z 100-1500 with a scan time of 1.9 sec. MS³ settings were identical to MS² except that the collision energy was 50-80 V and declustering potential was 50-80 V.

1.1.9 LC-MS/MS Analysis of TGFβ1 Incubated with SF Following Electrophoresis and Tryptic Digestion

1 μg aliquots of the TGFβ1 protein, supplied with bovine serum albumin as carrier, were incubated with either DMSO or 1.2 μmoles of SF for 30 minutes at 37° C. and run onto denaturing 4-12% Bis-Tris NuPAGE gels (Invitrogen, http://www.invitrogen.com). Bands were excised and digested with trypsin (Promega, http://www.promega.com/) after reduction with dithiothreitol (DTT) and alklyation with iodoacetamide (Sigma-Aldrich, http://www.sigmaaldrich.com/). Extracted peptides were lyophilized and re-dissolved in 1% acetonitrile, 0.1% formic acid for analysis by mass spectrometry. LC-MS/MS analysis was performed using a LTQ mass spectrometer (Thermo Electron Corporation, http://www.thermo.com/) and a nanoflow-HPLC system (Surveyor, Thermo Electron). Peptides were applied to a precolumn (C18 pepmap100, LC Packings, http://www.lcpackings.com/) connected to a self-packed C18 8-cm analytical column (BioBasic resin ThermoElectron; Picotip 75 μm id, 15 μm tip, New Objective, http://www.newobjective.com/). Peptides were eluted by a gradient of 2 to 30% acetonitrile in 0.1% formic acid over 40 min at a flow rate of approximately 250 nL min⁻¹. Data-dependent acquisition of MS/MS consisted of selection of the five most abundant ions in each cycle: MS mass-to-charge ratio (m/z) 300 to 2000, minimum signal 1000, collision energy 25, 5 repeat hits, 300 sec exclusion. In all cases the mass spectrometer was operated in positive ion mode with a nano-spray source and a capillary temperature of 200° C., no sheath gas was employed; the source voltage and focusing voltages were optimized for the transmission of angiotensin. Raw data were processed using BioWorks 3.3 (Thermo Electron Corporation). Searches were performed with Mascot (Matrix Science, http://www.matrixscience.com/) against SPtrEMBL (4719335 sequences) restricted by taxonomy to Homo sapiens (68982 sequences), oxidized methionine and carbamidomethyl cysteine residues were allowed as variable modifications as was putative SF. The error tolerance of the parent ion was ±1.2 Da and the fragment mass tolerance was ±0.6 Da, one missed cleavage was permitted. Error tolerant searches in Mascot against TGFβ were routinely performed and extracted ion chromatograms and manual inspection of spectra were prepared using Qual Browser and BioWorks 3.3 (Thermo Electron Corporation).

### 1.1.10 Luciferase Reporter Gene Assay

NIH 3T3 cells stably transfected with a CAGA12-luc plasmid, which responds to Smad activation (Dennler et al. (1998) Embo J 17: 3091-3100), were cultured in DMEM supplemented with 10% fetal calf serum (FCS), 1% penicillin, 1% streptomycin, 1% L-glutamine and 0.4 mg/ml geneticin. Cells were seeded into complete growth medium in a six-well tissue culture dish for 24 h, after which the medium was replaced with low serum medium (0.5% FCS) containing one of three treatments:

(1) TGFβ1 (to achieve a final concentration of 2 ng ml⁻¹) in PBS buffer,

(2) (TGFβ1+10 mM DTT in PBS buffer

(3) TGFβ1+2 μM SF in PBS buffer.

To simulate SF pharmacokinetics, all test samples were incubated at 37° C. for 30 minutes prior to dialysis, performed in PBS buffer for 4 hours using Slide-A-Lyzer Dialysis Cassettes MWCO 3.5K (PIERCE, http://www.piercenet.com/). Dialysis reduced SF concentration to 34 nM. As additional controls, cells were treated with PBS without TGFβ1 and PBS with SF (34 nM). The luciferase activity was determined 16 h following treatment using the Luciferase Reporter Gene assay (Roche Applied Science, http://www.roche.com/) in a Perkin Elmer Wallac Victor 2 1420 multilabel counter plate reader (http://las.perkinelmer.com/). Briefly, cells were washed twice with PBS and lysed in cell lysis buffer supplied with the assay. Chemiluminescence was immediately quantified following the addition of luciferase assay substrate. Luciferase values were normalized to protein concentration quantified using the BCA assay (Sigma-Aldrich, http://www.sigmaaldrich.com/). The experiment was repeated four times, with three replicates of each treatment per experiment. Statistical analysis was performed using 1-way ANOVA with the statistical software, R (http://www.R-project.org).

### 1.2 Results

### 1.2.1 Comparison of Gene Expression of Benign and Malignant TURP Tissue Samples

The inventors compared global gene expression profiles in surgically resected benign and malignant prostate TURP tissue using RNA extracted from heterogeneous tissue (such as we intended to use in the intervention study). Unsupervised clustering distinguished unambiguously the benign and malignant samples (data not shown). Pathway analyses for genes that were significantly different between the two groups were undertaken with the use of GenMapp software, and identified pathways that are frequently reported to be perturbed during carcinogenesis (Tables 3a and 4a). To validate further our methods of data analysis and to determine whether microarray data from gross heterogeneous tissue are comparable to data generated from LCD epithelial cells, we analyzed independent data sets of LCD epithelial cells (GEO Accession: GDS1439) from benign, localized and metastatic prostate cancer. We used our benign and malignant samples as a training set for linear discriminant analyses (LDA) and the independent data as test sets, and found that the LDA model correctly distinguished the benign, localized and metastatic LCD epithelial cell samples (FIG. 2). Thus, this preliminary study provides validation for our approach to the statistical analyses of array data.

### 1.2.2 Variation in Plasma PSA Levels

PSA levels prior to the intervention were in similar range to that previously reported for men of an equivalent age range diagnosed with HGPIN [25]. There was no significant association with GSTM1 genotype, and no consistent changes in PSA levels after six or 12 months within either arm of the intervention study (Table 2).

TABLE 2
Volunteer characteristics and plasma PSA levels.

| | | | PSA (ng/ml) | | |
|---|---|---|---|---|---|
| Age | BMI | GSTM1 | Pre-intervention | 6-month | 12-month |

Broccoli intervention

| 68 | 29 | null | 4.6 | 5.4 | 5.3 |
|---|---|---|---|---|---|
| 68 | 27 | null | 3.1 | 3.2 | 2.9 |
| 64 | 26 | null | 9.4 | 3.5 | 2.9 |
| 63 | 31 | null | 6.5 | 7.9 | 7.2 |
| 66 | 28 | null | 0.9 | 1.3 | 0.9 |
| Mean (sd) | | | 4.9 (3.25) | 4.3 (2.50) | 3.84 (2.44) |
| 57 | 27 | positive | 5.5 | 5.5 | 5.7 |
| 66 | 30 | positive | 13.6 | 16.8 | 13.4 |
| 69 | 27 | positive | 6.9 | 3.8 | 3.7 |
| 62 | 23 | positive | 2.2 | 2.2 | 1.9 |
| 59 | 29 | positive | 10.8 | 10.4 | 11.2 |
| 68 | 25 | positive | 9.7 | 12.5 | 7.2 |
| 64 | 32 | positive | 6.4 | 6.1 | 6.6 |
| 63 | 27 | positive | 7.9 | 9 | 10.8 |

| | | | |
|---|---|---|---|
| Mean (sd) | 7.9 (3.5) | 8.3 (4.85) | 7.56 (3.95) |

Peas intervention

| 70 | 28 | null | 4.1 | 4.2 | 4.4 |
|---|---|---|---|---|---|
| 65 | 24 | null | 7.5 | 9.3 | 8.2 |
| 59 | 24 | null | 9.3 | 10.8 | 8 |
| 61 | 35 | null | 1.1 | 1.1 | 1.1 |
| 57 | 26 | null | 5.5 | 5.4 | 5.2 |
| Mean (sd) | | | 5.5 (3.15) | 6.2 (3.92) | 5.4 (2.92) |
| 70 | 23 | positive | 8.9 | 5.2 | N/A* |
| 61 | 29 | positive | 2.3 | 2.2 | 2.5 |
| 57 | 30 | positive | 3.5 | 5.5 | 4.9 |

| | | | |
|---|---|---|---|
| Mean (sd) | 4.9 (3.52) | 4.3 (1.82) | 3.7 (1.70) |

One of the 22 volunteers was diagnosed with prostatic adenocarcinoma at the study baseline biopsy (pre-intervention) and was removed from the study.
*This volunteer developed prostatic adenocarcinoma six months into the intervention and was removed from the study.

TABLE 3
Pathway analyses of prostate biopsy tissue.

| | | |
|---|---|---|
| | Genes changed/ | Adjusted |
| Pathway | Genes on MAPP | P-value* |

a. Benign compared with malignant TURP tissue

| | | |
|---|---|---|
| Focal adhesion | 57/187 | <0.001 |
| TGFβ receptor | 47/151 | 0.002 |
| Circadian exercise | 20/48 | 0.006 |
| Fatty acid metabolism | 24/80 | 0.012 |
| Prostaglandin synthesis regulation | 14/31 | 0.026 |
| Actin binding | 53/213 | 0.028 |
| GPCRs Class A rhodopsin-like | 15/262 | 0.05 |

b. GSTM1 positives compared with GSTM1 nulls post
six month broccoli intervention

| | | |
|---|---|---|
| EGFR1 | 76/177 | <0.001 |
| Adipogenesis | 52/130 | 0.026 |
| TGF-beta receptor | 58/151 | 0.039 |

c. Paired samples pre and post 12 month dietary intervention

| | | |
|---|---|---|
| 0-12 month peas | | |
| Androgen receptor | 18/112 | 0.042 |
| 0-12 month broccoli** | | |
| mRNA processing | 40/125 | <0.001 |
| Androgen receptor | 33/112 | <0.001 |
| TGFβ receptor | 39/151 | 0.004 |
| Insulin signalling | 38/159 | 0.014 |
| Delta-notch | 24/85 | 0.019 |
| Wnt signalling | 28/109 | 0.02 |
| EGFR1 | 40/177 | 0.02 |
| IL-2 | 21/76 | 0.036 |

EGFR, epidermal growth factor receptor;
GPCRs, G-protein coupled receptors;
IL-2, interleukin 2;
TGFβ, transforming growth factor beta;
TURP, transurethral resection of the prostate;
Wnt, wingless-type MMTV integration site.
Pathways in GenMAPP that are enriched in the gene lists that differentiate groups are shown. Only pathways with adjusted P values ≦0.05 are shown.
Also, the number of genes changing between groups that belong to these pathways is shown alongside the total number of genes that constitute the pathway.
Pathway analysis was performed on gene lists generated in dChip that were statistically significant (P ≦ 0.05, Welch modified two-sample paired or unpaired t-test) between the two groups.
No fold cutoff was used. For details on gene lists see Table 3.
*P-values were calculated in GenMAPP using a non-parametric statistic based on 2000 permutations of the data and further adjusted for multiple testing by Westfall-Young adjustment.
**GSTM1 positive volunteers, n = 4.

### 1.2.3 Differences in Global Gene Expression Between GSTM1 Positive and Null Individuals

The inventors initially genotyped the resected TURP tissue samples and compared gene expression profiles between GSTM1 positive and null genotypes within the benign and malignant samples. They found few differences between genotypes, with similar high median false discovery rates (FIG. 3a, Table 4b). Likewise, the inventors compared gene expression profiles obtained from needle biopsies of the prostate from GSTM1 positive and null men who had previously been diagnosed with HGPIN and found few differences.

TABLE 4
Differentially expressed probes in prostate tissue.

| | | | |
|---|---|---|---|
| Fold | | | |
| change | P < 0.05* | P < 0.005* | P < 0.0005* |

(a) Differences between benign and malignant TURP tissue

| | | | | |
|---|---|---|---|---|
| Benign v Malignant | >1.0 | 3810 (353) | 683 (7) | 124 (0) |
| | >1.5 | 1081 (59) | 400 (2) | 104 (0) |
| | >2.0 | 277 (7) | 140 (0) | 54 (0) |

(b) Differences between GSTM1 positive and null genotypes

| | | | | |
|---|---|---|---|---|
| Benign (TURP) | >1.0 | 661 (538) | 19 (17) | 0 (0) |
| | >1.5 | 160 (186) | 7 (14) | 0 (0) |
| | >2.0 | 50 (40) | 1 (3) | 0 (0) |
| Malignant (TURP) | >1.0 | 686 (431) | 8 (13) | 0 (0) |
| | >1.5 | 244 (152) | 4 (8) | 0 (0) |
| | >2.0 | 44 (33) | 1 (3) | 0 (0) |
| Pre-intervention | >1.0 | 730 (484) | 26 (9) | 0 (0) |
| | >1.5 | 252 (79) | 16 (3) | 0 (0) |
| | >2.0 | 43 (9) | 8 (1) | 0 (0) |
| 6 month broccoli | >1.0 | 7976 (351) | 434 (4) | 17 (0) |
| | >1.5 | 2790 (91) | 268 (2) | 14 (0) |
| | >2.0 | 316 (8) | 31 (0) | 1 (0) |
| 6 month peas | >1.0 | 220 (220) | 6 (4) | 0 (0) |
| | >1.5 | 33 (41) | 5 (3) | 0 (0) |
| | >2.0 | 5 (10) | 1 (1) | 0 (0) |

(c) Differences between paired samples

| | | | | |
|---|---|---|---|---|
| 0-12 months | >1.0 | 2857 (96) | 151 (0) | 1 (0) |
| broccoli | >1.5 | 1243 (12) | 141 (0) | 1 (0) |
| | >2.0 | 213 (0) | 62 (0) | 1 (0) |
| 0-12 months peas | >1.0 | 1199 (42) | 19 (0) | 0 (0) |
| | >1.5 | 495 (18) | 19 (0) | 0 (0) |
| | >2.0 | 81 (1) | 4 (0) | 0 (0) |

Table 4: Probe numbers that have satisfied the comparison criteria of fold change and P-value cutoffs are shown. Numbers in parentheses represent the median false discovery rate calculated in dChip after 100 permutations of the samples.
*P-values were calculated in dChip by a Welch modified two-sample t-test. n = 18 for Benign, n = 14 for Malignant; n = 4 for GSTM1(+) Benign, n = 14 for GSTM1(−) Benign; n = 5 for GSTM1(+) Malignant, n = 9 for GSTM1(−) Malignant; n = 7 for GSTM1(+) Pre-intervention, n = 3 for GSTM1(−) Pre-intervention; n = 6 for GSTM1(+) 6-month broccoli, n = 5 for GSTM1(−) 6-month broccoli; n = 3 for GSTM1(+) 6-month pea and n = 5 for GSTM1(−) 6-month pea intervention.
**P-values were calculated in dChip by a Welch modified two-sample paired t-test, n = 4 for each of the diet interventions.

They then compared gene expression profiles between GSTM1 genotypes in needle biopsy tissue of twenty-one men who had been recruited into the dietary intervention study. Eight of the men within this study had been asked to consume 400 g of steamed frozen peas per week, and the other thirteen were requested to consume 400 g of steamed frozen broccoli per week, but otherwise to consume their normal diet. Diet was assessed with seven-day diet diaries prior to the intervention and after six months. No significant differences were found in diet components, apart from the consumption of broccoli and peas (Table 5). The inventors found many differences in the prostate gene expression between GSTM1 positive and null men who had been on the broccoli diet for six months, but few, if any, differences in gene expression between GSTM1 positive and null men who had been on the pea diet (FIG. 3a, Table 4b). To investigate the potential consequences of the differences in gene expression between GSTM1 genotypes following the broccoli-rich diet, they analyzed these data via GenMapp. Three pathways, EGF receptor, adipogenesis and TGFβ receptor, were identified in which genes occurred at a higher frequency than they would by chance (Table 3b).

TABLE 5
Dietary analysis of average daily intakes of nutrients.

| | | | |
|---|---|---|---|
| Variable | Baseline | 6 months | P-value* |

Pea-rich diet (n = 7)

| | | | | | |
|---|---|---|---|---|---|
| Fat (g) | 93.76 | (32.97) | 90.99 | (25.83) | 0.831 |
| Protein (g) | 87.50 | (15.15) | 95.77 | (33.31) | 0.526 |
| CHO (g) | 240.04 | (79.85) | 242.94 | (72.97) | 0.872 |
| Energy (KJ) | 9081.88 | (2480.80) | 9506.00 | (2405.38) | 0.738 |
| Alcohol (g) | 12.65 | (9.87) | 25.07 | (35.42) | 0.415 |
| Cholesterol (mg) | 353.57 | (74.43) | 345.29 | (201.06) | 0.930 |
| Vitamin C (mg) | 81.00 | (66.05) | 79.43 | (62.58) | 0.846 |
| Vitamin E (mg) | 8.24 | (5.64) | 7.15 | (3.60) | 0.395 |
| Vitamin D (μg) | 3.77 | (3.25) | 3.84 | (1.76) | 0.949 |
| β-Carotene (mg) | 1.94 | (1.57) | 3.22 | (2.29) | 0.184 |
| Folate (μg) | 261.00 | (89.36) | 332.86 | (214.50) | 0.489 |
| Iron (mg) | 11.73 | (4.36) | 13.32 | (4.92) | 0.555 |
| Selenium (μg) | 50.14 | (13.01) | 49.14 | (23.08) | 0.923 |
| Peas (g) | 8.57 | (10.23) | 57.41 | (18.86) | 0.001 |
| Broccoli (g) | 18.49 | (30.89) | 9.90 | (13.61) | 0.431 |
| Estimated GSL | 9.36 | (15.63) | 5.01 | (6.88) | 0.431 |
| (μmol) | | | | | |

Broccoli-rich diet (n = 11)

| | | | | | |
|---|---|---|---|---|---|
| Fat (g) | 90.93 | (29.27) | 91.57 | (33.70) | 0.929 |
| Protein (g) | 96.99 | (20.02) | 96.97 | (21.26) | 0.996 |
| CHO (g) | 276.28 | (76.03) | 296.18 | (72.99) | 0.305 |
| Energy (KJ) | 9633.45 | (2311.35) | 9980.73 | (2286.62) | 0.488 |
| Alcohol (g) | 9.75 | (7.20) | 10.48 | (9.70) | 0.841 |
| Cholesterol (mg) | 337.27 | (168.29) | 298.46 | (123.99) | 0.211 |
| Vitamin C (mg) | 262.55 | (175.83) | 303.00 | (188.52) | 0.590 |
| Vitamin E (mg) | 11.31 | (5.73) | 11.14 | (4.82) | 0.924 |
| Vitamin D (μg) | 5.22 | (3.17) | 3.65 | (1.08) | 0.076 |
| Beta Carotene | 4.07 | (3.01) | 3.63 | (2.53) | 0.667 |
| (mg) | | | | | |
| Folate (μg) | 477.82 | (188.20) | 491.36 | (193.47) | 0.762 |
| Iron (mg) | 14.29 | (2.09) | 14.09 | (2.25) | 0.916 |
| Selenium (μg) | 77.09 | (29.89) | 68.82 | (26.22) | 0.304 |
| Peas (g) | 4.16 | (5.51) | 7.60 | (8.46) | 0.227 |
| Broccoli (g) | 25.89 | (24.49) | 55.84 | (7.71) | 0.002 |
| Estimated GSL | 13.10 | (12.39) | 79.30 | (10.94) | <0.0001 |
| μmol | | | | | |

Variables shown are given in mean (sd) units per day. GSL refers to the glucosinolate precursors of sulforaphane and iberin (ie 4-methylsulphinylbutyl and 3-methylsulphinylpropyl glucosinolate) respectively. Similar analysis between GSTM1 positive and null individuals showed no difference in dietary intakes after 6 months within either broccoli-rich or pea-rich intervention.
*P-values were calculated in Minitab using a paired t-test.

1.2.5 Changes in Gene Expression Before and after the Dietary Intervention

Paired t-tests were used to identify genes that had changed in expression between 0 and 6 months and 0 and 12 months in biopsy samples from individuals within each arm of the intervention to quantify changes in expression with time. Within the broccoli arm, analyses were restricted to GSTM1 positive individuals. The inventors found after both 6 months and 12 months there were more changes in expression within the broccoli-rich arm than the pea-rich arm (FIG. 3b, Table 4c). Pathway analyses with genes that changed in expression between 0 and 12 months identified changes only in the androgen receptor pathway in the pea-rich arm, while in the broccoli-rich arm androgen receptor pathway was identified, along with several other signalling pathways, including insulin signalling, TGFβ and EGF receptor pathways (Table 2c). Analyses with genes that changed in expression between 0 and 6 months in the broccoli arm also identified changes in TGFβ receptor pathway (adjusted P=0.001), insulin signalling (adjusted P=0.035) and EGF receptor signalling (adjusted P=0.068).

Thus, evidence for the effect of broccoli consumption on modulation of TGFβ and EGF signalling has been obtained in two independent analyses: Firstly, the comparison of gene expression profiles of GSTM1 positive and null individuals who had consumed the broccoli-rich diet for six months, and, secondly, the paired analyses of gene expression profiles from biopsies obtained at 0 and 12 months from GSTM1 positive individuals who had consumed the broccoli-rich diet. It is important to note that these analyses do not share any array data sets.

### 1.2.6 Chemical Interactions Between TGFβ1, Insulin and EGF Peptides and Broccoli Isothiocyanates

Having demonstrated that broccoli consumption modulates several cell signalling pathways, the inventors sought a mechanistic explanation. Incubation of insulin, EGF and TGFβ1 peptides with the isothiocyanates SF or IB in PBS pH 7.4 at 37° C. for a period of 0.5 to 24 hours gave consistent evidence of the formation of a covalently bound conjugate of the respective peptide and the ITC. This was further investigated for physiological relevance by performing the same incubations in human plasma depleted of high MW proteins. LC-MS/MS analysis showed the appearance of one or more additional LC-MS peaks when SF or IB were incubated with the peptides. For example, in FIG. 4 an extracted ion chromatogram (m/z 1183.9, corresponding to insulin-SF MH₅⁵⁺) shows the appearance of two insulin-SF conjugates compared with the control incubation. MS² analysis of these peaks (FIG. 5) confirmed the presence of two diagnostic fragment ions at m/z 235 and m/z 325 corresponding to the addition of SF to the two N-terminal amino acids of insulin Gly-SF and Phe-SF. Similar results were obtained to identify Gly-IB (m/z 221) and Phe-IB (m/z 311) from the incubation (data not shown). Comparable evidence was obtained for the formation of EGF conjugates with SF in human plasma corresponding to the addition of SF to the N-terminal asparagines residue (m/z 309) of EGF.

To provide additional information of modifications to TGFβ1, the inventors adopted a complementary approach. 1 μg aliquots of the protein were incubated with either DMSO or 1.2 μmoles of SF for 30 minutes at 37° C. and separated by SDS-PAGE electrophoresis. Bands were excised and digested with trypsin before analysis by LC-MS/MS. TGFβ1 was robustly identified in bands of 25 kDa corresponding to the active dimer. The N-terminal peptide ALDTNYCFSSTEK (SEQ ID No. 7) was identified from parent ion m/z 768.5 in both DMSO (control) and SF-treated samples (FIG. 6). A precursor ion m/z 877.2 was observed only in SF treated samples. MS/MS analysis of both precursor ions revealed a strong series of fragment peaks that were common to both (FIG. 7) precursor ions. These fragmentation patterns are consistent with the unmodified y ion series for the peptide ALDTNYCFSSTEK (including carbamidomethyl cysteine +57) and a b ion series shifted by 217.4±0.8 Da in the SF-treated sample. These results strongly support an N-terminal modification to TGFβ1 by SF. Addition of SF would result in a mass addition of 177, as observed with LC-MS analyses of intact TGFβ1, as described above. It is highly likely that the addition of 217, as opposed to 177, is due to subsequent reaction of the thiourea with iodoacetamide, added to the reaction mixture to alkylate reduced disulphide linkages, to result in a mixture of isomeric carbamimidoylsulfanylacetamides, which undergo cyclisation and loss of NH₃ to give the corresponding iminothiazolidinones.

1.2.7 Enhancement of TGFβ1 Signalling after Pre-Incubation with Sulforaphane

The inventors sought to assess whether SF modification of extracellular signalling proteins had functional consequences. We focussed on TGFβ1 signalling due to its profound role in maintaining tissue homoeostasis through controlling cell proliferation and behaviour. TGFβ1-induced Smad-mediated transcription was quantified in NIH3T3 cells stably transfected with a CAGA12-luc plasmid, in which luciferase activity can be measured upon activation of Smad proteins. Exposure of cells to TGFβ1 induced luciferase activity as expected. When cells were exposed to TGFβ1 that had been pre-incubated with physiologically appropriate concentrations of SF (2 μM) for 30 minutes followed by dialysis, to simulate SF plasma pharmacokinetics, there was an increase in Smad-mediated transcription compared to exposure to TGFβ1 alone (FIG. 8). Exposure of cells to the residual SF (34 nM) did not result in enhanced transcription suggesting that SF induces Smad activation indirectly, consistent with our previous observation that SF binds to the ligand itself. It is also conceivable that SF may interact with the extracellular domain of the receptor to alter downstream signalling.

### 1.3 Discussion

This is the first dietary intervention study to analyse global gene expression profiles within a target tissue before and after a 12 month intervention, and to stratify gene expression profiles by genotype. While the inventors did not observe any consistent changes in plasma PSA levels over the 12 month period of the intervention, they were able to quantify extensive changes in gene expression.

There was little evidence to support potential mechanisms derived from animal and cell models to explain the observational data that consuming broccoli may reduce risk of cancer. However, to their inventors surprise, generated considerable evidence for the perturbation of several signalling pathways that are associated with inflammation (Table 2b and c). The inventors believe that the net effect of perturbation of these pathways may reduce the risk of cell proliferation, and maintain cell and tissue homoeostasis. However, whilst quantification of gene expression and pathway analyses provides information concerning which pathways may be modified by time or diet, it can provide little information about the precise nature of how these pathways are perturbed. This required further analysis of mRNA and protein turnover, and post translational protein modifications such as phosphorylation, associated with components of the signal transduction pathway and downstream targets. It was of considerable interest that broccoli intervention was associated with perturbation of TGFβ1, EGF and insulin signalling, each of which has been associated with prostate carcinogenesis, in addition to carcinogenesis at other sites and also inflammation (e.g. associated with myocardial infarction). It is noteworthy that broccoli consumption was also associated with alterations in mRNA processing, and this is being further explored.

The inventors believe that the anti-inflammatory bioactive products derived from broccoli are the isothiocyanates, sulforaphane and iberin. These have been shown to have a multitude of biological activities in cell models consistent with anticarcinogenic activity. However, these studies largely involve exposing cells to concentrations of SF and IB far in excess of those which occur transiently in the plasma after broccoli consumption, and are mediated by the intracellular activity of the ITCs by, for example, perturbing intracellular redox status, depletion of glutathione and perturbation of the Keap1-Nfr2 complex. The inventors question whether these processes would occur in vivo with levels of ITCs that would be derived from the diet. Any of the ITCs entering cells would immediately be inactivated through conjugation with glutathione that would be present in relatively high concentration. Thus, they explored whether the biological activity of ITCs may be mediated through their chemical interaction with signalling peptides within the extracellular environment of the plasma, which has a low glutathione concentration. The inventors demonstrated that ITCs readily form thioureas with signalling proteins in the plasma through covalently bonding with the N-terminal residue. It is likely that ITCs chemically react with other plasma proteins and a global analysis of plasma protein modifications by ITCs is warranted. It is also possible that other types of chemical modification of plasma proteins by ITCs may occur, such as covalent bonding through cysteine and lysine residues.

Previous studies have shown that isothiocyanate-derived thioureas modify the physicochemical and enzymatic properties of the parental proteins. Thus, they inventors believe it is possible that the perturbation of signalling pathways in the prostate is mediated by protein modifications that occur in the extracellular environment. This study provides evidence for this hypothesis by demonstrating that pre incubation of TGFβ1 with a physiological appropriate concentration of SF (2 μM for 30 minutes), followed by dialysis for 4 h to simulate SF pharmacokinetics, results in enhanced Smad-mediated transcription. As TGFβ1/Smad-mediated transcription inhibits cell proliferation in non-transformed cells, the enhancement of Smad-mediated transformation by SF would be consistent with the anticarcinogenic activity of broccoli, in addition to reduced risk of myocardial infarction and, as realised for the first time in this study, represents a mechanism for inhibiting the inflammatory reaction. It will therefore be appreciated that this study shows that a plant extract rich in ITCs is able to activate the Smad pathway and as such ITCs represent anti-inflammatory agents.

Perturbation of signalling pathways is additionally determined by GSTM1 genotype. The interaction between diet and GSTM1 on gene expression may partially explain the contradictory results from those case control studies which lack dietary assessment and which have or have not associated the GSTM1 null genotype with enhanced risk of prostate cancer. GSTM1 enzyme activity catalyses both the formation and the cleavage of SF-glutathione conjugates. The inventors suggest that following transport into the plasma from enterocytes, GSTM1 activity (originating either from hepatic cell turnover or leakage from peripheral lymphocytes) catalyses the cleavage of the SF-glutathione conjugate within the low glutathione environment of the plasma to determine the extent of free SF that is available for protein modification, as discussed above, and which is not excreted via mercapturic acid metabolism (FIG. 1). Thus low levels of SF, as would be expected from normal dietary consumption of broccoli, may lead to subtle changes in cell signalling, which, over time, result in profound changes in gene expression. In this manner, consuming one portion of broccoli per week if one is GSTM1 positive, or more if one is GSTM1 null, may contribute to a reduction in cancer risk and also a reduction in the risk of developing an inflammatory condition.

In addition to the insight this study provides to the effect of broccoli consumption on gene expression, the inventors consider that this study may have broader implications. Their knowledge of the signalling pathways that are modulated by ITC rich plant extracts made them realise that other dietary phytochemicals, such as polyphenolic derivatives, could also chemically interact with signalling peptides in the plasma, in a similar manner to the suggested mechanism of action of isothiocyanates. Further work (see the following examples) established that ITCs and ITC rich plant extracts are indeed useful for preventing or treating medical conditions characterised by having an inflammatory component. Further ITC may be combined with extracts rich in polyphenolics (as defined by the seventh aspect of the invention) to provide compositions with additive and synergistic effects that may be used to great effect to prevent the development or treat conditions according to the invention.

### EXAMPLE 2

A range of experiments were conducted to demonstrate the way in which interactions occur between ITCs and plant extracts enriched with ITCs. The functional effects of these interactions on signalling in inflammatory pathways, and the overall effects of the interactions on inflammatory markers were demonstrated.

The protocols and methods utilised in Example 1 were employed in the following experiments (except where indicated otherwise).

### Experiment 1

FIGS. 9-13 illustrate that ITCs can form conjugates with inflammatory signalling peptides. Incubation of ITCs with TGF beta and EGF results in an N-terminal modification of the peptides to form thioureas.

Data is presented to show conjugate formation between signalling molecules TGF beta and EGF and a range of ITCs, representing representative of ITC found naturally.

### Experiment 2

An example of the functional consequences of the interactions of ITCs with TGFβ1 was demonstrated for two cell types. TGFβ1 acts an anti-inflammatory cytokine. It induces phosphorylation of smad proteins that translocate to the nucleus and induce gene expression associated with anti-inflammatory activity. The inventors have found that ITCs will not induce pSmad 2 without TGFβ1 but, when combined with the growth factor, cause a significant induction of pSmad. Therefore ITCs act as anti-inflammatory mediators.

In table 6 it can be seen that co-exposure to TGF β1 and SF enhances expression of psamd2 in the PC3 cell line, compared to the effects TGFβ1 alone. Likewise, in Table 7 it can be seen that co-exposure to TGFβ1 and Erucin (ER) also enhances expression of psmad2 in the A549 cell line. The data is further presented in FIGS. 14 and 15 respectively.

TABLE 6
Quantification of pSmad2 protein in PC3 cells. Experiments was
performed in triplicate. Data shown in counts/mm².
Loading of total protein was normalised to GAPDH.

| | PSmad2 | GAPDH | Normalised to GAPDH |
|---|---|---|---|
| TGFβ1 | 6154 | 59200 | 0.1039 |
| TGFβ1 | 10910 | 63344 | 0.1722 |
| TGFβ1 | 7089 | 50678 | 0.1398 |
| TGFβ1 + 2 μM SF | 8179 | 46135 | 0.1772 |
| TGFβ1 + 2 μM SF | 12578 | 44800 | 0.2807 |
| TGFβ1 + 2 μM SF | 11881 | 38437 | 0.3091 |
| TGFβ1 + 10 μM SF | 13595 | 52243 | 0.2602 |
| TGFβ1 + 10 μM SF | 16870 | 60035 | 0.2810 |
| TGFβ1 + 10 μM SF | 15049 | 48468 | 0.3104 |

TABLE 7
Quantification of pSmad2 protein in A549 cells.
Data shown in counts/mm².
Equal loading of protein was normalised to GAPDH.

| | pSmad2 | GAPDH | Normalised to GAPDH |
|---|---|---|---|
| Control | 46 | 14201 | 0.003 |
| TGFβ1 | 1570 | 11893 | 0.1320 |
| TGFβ1 + 2 μM ER | 3079 | 4911 | 0.6269 |
| TGFβ1 + 10 μM ER | 3916 | 11936 | 0.3280 |

### Experiment 3

A further example of the functional consequences of interactions between ITCs and signalling peptides is given, showing that incubation of ITCs with EGF can suppress EGF signalling in BPH cells, a model of hyperplastic prostatic tissue. EGF binds to and phosphorylates the EGF receptor which activates the down stream signalling pathway. FIG. 16 presents data that shows that pre-incubation of EGF with 4-methylsulphinylbutyl ITC under conditions known to cause peptide modification reduces the amount of phosphorylated receptor compared to EGF alone. This will result in inhibition of the EGF signalling pathway and illustrates further anti-inflammatory consequences of ITC treatment.

### EXAMPLE 3

Experiments were conducted to demonstrate that ITCs, and plant extracts enriched with ITCs, have an anti-inflammatory effect by reducing TNF-α induced IL-6 production.

Further experiments were conducted to demonstrate that the effect of ITCs was increased when ITCs are mixed with procyanidins according to the seventh aspect of the invention.

Results are presented in FIG. 17 using Erucin (ER) as an ITC and a mixed procyanidin extract (GE). Both ER and GE show efficacy in suppressing TNF-α induced IL-6 expression by HUVEC cells. Surprisingly a combination of ER and GE caused a reduction in IL-6 generation that was larger than that seen for either test mix when tested individually at the same concentrations.

The extract GE used in this experiment was prepared as follows:

40 mg powdered grape skin/seed extract was dissolved in 1 ml 70% MeOH, heated to 70° C. for 20 minutes, centrifuged at 4500 rpm for 15 minutes and filtered to 0.45μ, giving a solution with procyanidin concentration of 11.016 mg/ml. The filtered solution was diluted 1/100 with PBS before use to give 110.16 μg/ml stock. Mixtures were prepared to contain final concentrations of 100 μmol/L ITC and 20 mg/L procyanidin.

Overall Examples 1-3 illustrate that ITCs in general, and particularly those from rocket species, can modify multiple inflammatory pathways. Significantly their anti-inflammatory effects can be enhanced by combination with procyanidins, leading to an anti-inflammatory formulation with multiple biological targets.

### EXAMPLE 4

### Production of an ITC-Rich Extract for Use According to the Invention

### Preparation 1

(i) Fresh leaves from young rocket plants (28-42 days) were dried (either by (a) air drying or (b) by snap freezing and freeze drying). The dried leaves were then milled to a fine powder so that particle size was <100 microns.

(ii) A suspension of this powder was prepared by mixing powder with water at pH 7 at 20-30° C. to give a mixture with a minimum of 10% solids and a maximum 50% solids.

(iii) An extract may then be prepare using a counter-current extractor, equipped with a vapour trap to retain volatiles extracted into solution, or a Soxhlet-type extractor operating under reduced pressure and fitted with a reflux condenser. Extraction should proceed for a minimum of 1 hour at 20-30° C. and/or until a minimum of 50%, and preferably >70%, of the native glucosinolates from the rocket has been converted to ITCs by the action of native enzymes.

(iv) Once extraction is complete, solids can be removed from the suspension by centrifugal separation or decanting. The ITC-rich supernatant can be deproteinated by chemical or enzymatic means, or by filtration (e.g. ultrafiltration), and concentrated by low-temperature high vacuum evaporation, or by removal of water by reverse osmosis.

(v) The final extract can be stored frozen as a liquid or spray-dried to give a powder, or encapsulated (e.g. in a fat matrix, or in a polysaccharide matrix, or in a polymer matrix) to enhance stability. The final extract should be standardised to contain between 10-100 μmol/L total ITCs

As an alternative to fresh leaves, young sprouts (up to 14 days) can be used as the starting material.

### Preparation 2

Seeds can be used as the starting material. In the case of seeds, air drying is sufficient preparation, and the dry seeds can then be crushed (for example using a sealed press) in the presence of water to give a high solids mash (between 75% and 90% solids). Crushing should proceed until a homogenous mash is formed with particle size not exceeding 250 micron; thereafter the extraction can proceed as described above (see (iii)-(v)).

It will be appreciated that a mixture of sprouts/leaves/seeds (i.e. preparations 1 and 2) may be used as the starting material, to ensure an ITC extract containing a wide range of structures is prepared. Leaves and sprouts contain higher levels of 4-mercaptobutyl GLS than seeds, which are higher in 4-methylthiobutyl GLS.

### EXAMPLE 5

### Production of a Glucosinolate Rich Extract (i.e. an ITC Precursor According to the Invention) for Use According to the Invention

Starting materials may be seeds, sprouts or leaves (preferably dried prior to extraction) as described in Example 4.

Prepare a suspension of dried, milled starting material in ethanolic solution (70%-85% ethanol), to give a mixture with minimum 10% solids, maximum 50% solids. The ethanol used should be food-grade. Heat in a reactor (preferably a counter-current continuous extractor or a Soxhlet-type extractor equipped with condensers to catch volatiles) at 70° C. for a minimum of 20 minutes or until between 70% and 90% of the native glucosinolates have been extracted into ethanolic solution. Remove solids from the suspension by centrifugal separation or decanting, preferably using explosion-proof conditions. Remove ethanol from the supernatant by, for example, evaporation under reduced pressure, or by reverse osmosis (using diafiltration) after first diluting the supernatant to <40% ethanol. The final solution should contain <5% ethanol.

This glucosinolate-rich solution can either be stored frozen, or can be spray-dried to give an ethanol-free powder. To convert the glucosinolates to ITCs, the glucosinolate-rich extract should be dissolved in water at pH 7 and 20-30° C., and the conversion should be carried out by adding myrosinase enzyme, either in purified form or as part of a crude rocket-seed/mustard-seed mash. The mixture should be incubated for between 1 and 7 hours, or until a minimum of 50%, and preferably >70%, of the native glucosinolates have been converted to ITCs. Solid material and protein should be removed from the ITC-rich solution by filtration (e.g. microfiltration or ultrafiltration), and the extract can then be concentrated as previously described.

### EXAMPLE 6

### Production of a Powder Mix for Use According to the Invention

2.0 g of freeze dried powder (Example 4 or 5) was mixed with 0.5 g powdered citric acid, 27.3 g of maltodextran and 0.2 g of a standard spray-dried mix of flavouring.

This mixture represents a free-flowing powder formulation (containing 2.0 g of plant extract) that is suitable for packaging in a sachet. The powder mix may be diluted to taste and drunk when required by a subject suffering from a condition with an inflammatory component.

### EXAMPLE 7

### Production of a Grape Drink for Use According to the Invention

Two drink products were formed comprising 0.02 g or 0.2 g of freeze-dried powder (prepared according to Example 4 or 5) dissolved in 100 mls of Grape juice (or alternatively with: (a) Grape juice concentrate and water; (b) a blend of fruit juices which may include grape juice).

The grape drink preparations may be consumed by a subject immediately, refrigerated for later consumption or sealed in a bottle or carton for a longer shelf life.

It will be appreciated that the grape juice will comprise polyphenols and drinks prepared according to this Example represent preferred compositions according to the seventh aspect of the invention.

The grape juice may be readily substituted with a palatable alternative (e.g. orange juice or the like) to form preferred compositions according to the first-sixth aspects of the invention.

### EXAMPLE 8

### Method for Preparing Grape Skin/Seed Extracts

Red or white grape skins, with their seeds and stalks, were used as a starting material for extraction.

Grapes skin/seed mixtures were air dried on a heated belt dryer. The dried starting material was then milled finely to produce a powder with particle size <250 micron. Preparation of high-polyphenol extracts, containing a high proportion of procyanidins, was carried out by continuous extraction using counter-current extractors, in ethanol/water mixtures. The extractants may be acidified by addition of, hydrochloric, citric or tartaric acids, so that the pH range is between 1.5 and 4, to improve recovery if a high proportion of grape skins is present. This is not always necessary, especially if the proportion of seeds is high. The extractants should contain between 45% and 65% ethanol, and may contain in addition up to 15% acetone.

Extraction may be carried out in a single pass, but preferably two or three sequential extraction stages may be employed to maximise recovery.

Once extraction is complete, solids are removed from the suspension by centrifugal separation or decanting. The procyanidin-rich supernatant can be deproteinated by chemical or enzymatic means, or by filtration (e.g. ultrafiltration), and concentrated by low-temperature high vacuum evaporation, or by removal of water by reverse osmosis.

The final extract can be stored frozen as a liquid or spray-dried to give a powder, or encapsulated (e.g. in a fat matrix, or in a polysaccharide matrix, or in a polymer matrix) to enhance stability. The final extract should be standardised to contain between 0.5-1.5 g/L procyanidins.

### EXAMPLE 9

### Encapsulated Mixes of ITCs and Procyandins

Encapsulation of the ITC-rich extracts of Example 4 or 5 and procyanidin-rich extracts of Example 8 is carried out by first preparing a solution of the extracts in ethanolic solution at a concentration of between 50% and 70% dry matter. The concentration of ethanol may be between 0% and 10%. The proportion of procyanidin extract to ITC extract may be 3:1 or 5:1 or 10:1.

The prepared solution should be mixed in equal volumes with a suitable encapsulant shell matrix. For example, a mixture of fats, or a solution of polysaccharides such as alginates, or a solution of polymeric material such as chitosan. The mixture should be thoroughly homogenised at a temperature not exceeding 90 C, and formed into particles by either spray drying, or by forming an aerosol and cooling, or by other known encapsulation techniques. Final particle size should not exceed 100 micron.

The resulting encapsulates may be either hard-shell or soft-shell, and should contain a minimum of 10% extract w/w, but preferably between 20% and 50% extract w/w.

### EXAMPLE 10

### Powder Mixes of ITCs and Procyandins

ITC-rich extracts of Example 4 or 5 (comprising 1%-10% ITCs) and procyanidin-rich extracts of Example 8 were made into powders (by spray-drying). The two powders were then combined such that the proportion of procyanidin extract to ITC extract was 3:1 or 5:1 or 10:1.

This powder mix represented another preferred composition which may be as an ingredient to be added to pharmaceutical products, nutraceutical products, drinks, foods and the like according to the seventh aspect of the invention.

1. A composition, comprising an inflammatory component comprising a therapeutically effective amount of an isothiocyanate (ITC).

2. The composition according to claim 1 wherein the ITC is derivable from a plant of the genus Brassica.

3. The composition according to claim 2 wherein the plant is a rocket spp.

4. The composition according to claim 1 wherein the ITC is at least one of sulforaphane (SF), iberin (IB) or erucin (ER-4-methylthiobutyl isothiocyanate).

5. The composition according to claim 1 wherein the ITC is within a plant extract enriched with ITC or a precursor of ITC.

6. The composition according to claim 5 wherein the plant extract is from a plant of the genus Brassica.

7. The composition according to claim 6 wherein the plant is a rocket spp.

8. The composition according to claim 1 further comprising another anti-inflammatory agent.

9. The composition according to claim 8 wherein the agent is an NSAID or corticosteroid.

10. The composition according to claim 8 wherein the agent is a polyphenol.

11. The composition according to claim 10 wherein the agent is a plant extract enriched in polyphenols.

12. The composition according to claim 10 wherein the polyphenol is a procyanadin.

13. The composition according to claim 10 wherein the polyphenol is a flavanoid.

14. The composition according to claim 13 wherein the flavanoid is flavan3ol.

15. The composition according to claim 10 wherein the polyphenol is derived from a fruit skin or seed.

16. The composition according to claim 10 wherein the polyphenol is derived from a Vinus spp.

17. A method of treating Rheumatoid Arthritis, comprising providing the composition of claim 1 and administering the composition to a subject to treat Rheumatoid Arthritis in the subject.

18. A method of treating Inflammatory Bowel Disease, comprising providing the composition of claim 1 and administering the composition to a subject to treat Inflammatory Bowel Disease in the subject.

19. A beverage comprising the composition of claim 1.

20. A nutritional product comprising the composition of claim 1.

21. A pharmaceutical product comprising the composition of claim 1.
